(19) United States
(12) Patent Application Publication
(10) Pub. No.: US 2004/0197833 A1
(43) Pub. Date: Oct. 7, 2004

(54) Method for the enrichment of target cells by use of CBDs

(75) Inventor: Martin Loessner, Zurich (CH)

Correspondence Address: Steven L. Highlander, FULBRIGHT & JAWORSKI L.L.P., 600 Congress Avenue, Suite 2400, Austin, TX, 78701, US

(73) Assignee: Danisco A/S, Copenhagen (DK)

(21) Appl. No.: 10/406,897

(22) Filed: Apr. 3, 2003

(51) Int. Cl. G01N 33/53; G01N 33/567; C12N 5/08

(52) U.S. Cl. 435/7.2; 435/366

(57) ABSTRACT

The present invention concerns a method for the enrichment of target cells by binding, wherein cell wall binding domains are used. Specifically, the present invention concerns a method for the specific recognition of target cells by binding wherein the CBDs are covalently bound to a solid phase and wherein said solid phase consists of beads, preferably magnetic latex beads. The invention also relates to the use of said method for detection, diagnosis, immobilisation or enrichment of cells. The invention furthermore relates to a reaction kit for a method as described above, wherein said kit comprises additionally to conventional detection means one or more CBDs. Finally, the present invention also relates to biochips, comprising CBDs, specifically biochips comprising two or more different CBDs in defined locations.

The present invention concerns a method for the enrichment of target cells by binding, wherein cell wall binding domains are used. Specifically, the present invention concerns a method for the specific recognition of target cells by binding wherein the CBDs are covalently bound to a solid phase and wherein said solid phase consists of beads, preferably magnetic latex beads. The invention also relates to the use of said method for detection, diagnosis, immobilisation or enrichment of cells. The invention furthermore relates to a reaction kit for a method as described above, wherein said kit comprises additionally to conventional detection means one or more CBDs. Finally, the present invention also relates to biochips, comprising CBDs, specifically biochips comprising two or more different CBDs in defined locations.

For the diagnosis and detection of specific agents in food, environmental, blood and other samples, it is increasingly of importance to provide reliable and quick detection means and methods, which can provide the technician or practitioner with results within the shortest possible time span and with a high rate of reliability.

To be able to achieve said goal, a multiplicity of methods and means has already been developed. However, these methods and means often suffer from either a lack of reliability, a lack of quick results due to often consuming and complicated working procedures or both.

One group of bacteria which so far cannot be detected in a satisfactory manner is the genus Listeria.

In the following, said genus will be introduced in more detail and the problems encountered with its diagnosis and detection will also be described. However, this description is only exemplary and non-limiting and the problems encountered with the diagnosis and detection of Listeria are encountered with a multiplicity of other bacteria and pathogens as well, and all conclusions that are drawn for Listeria apply mutatis mutandis for all equal problems as encountered with other pathogens as well.

In 1926 Murray et al., (Murray, E. G. D., R. A. Webb, M. B. R. Swann. 1926. A disease of rabbits characterized by large mononuclear leucocytosis caused by a hitherto undescribed bacillus Bacterium monocytogenes. J. Path. Bact. 29: 407-439) discovered a bacterium which was the cause of a high mortality rate in rabbits. The characteristic diagnosis included a drastic increase of monocytes. The name for this bacterium changed several times, however, finally, it was given the genus name Listeria in 1940 (Pirie, J. H. H. 1940, Listeria: Change of name for a genus of bacteria. Nature 145:264).

Although it was known since 1929 that the genus Listeria was also pathogenic for humans, only during the 1980's further scientific research was carried out on Listeria monocytogenes, which is the main human pathogen within the genus Listeria.

Rocourt showed in 1994 (Rocourt, J. 1994. Listeria monocytogenes: the state of the science. Dairy Food Environ. Sanit. 14:70-82) that there were outbreaks of listeriosis with lethality rates of up to 30%. Thus, it became clear how dangerous this pathogen was and is for humans. Specifically, subjects with a depressed immune system, pregnant women, unborn and new-born babies and older people are endangered by said bacteria. Listeria

can be encountered biquitously and are therefore easily included in food. Besides originally contaminated raw food, there is further danger from insufficient heating or re-contamination.

Classification of Listeria was difficult for a long period of time. Only with the advent of molecular biological methods like rRNA sequence analysis and DNA/DNA hybridisations, it became possible to recognise clearly the genus Listeria, which is divided into six species, namely L. monocytogenes sensu stricto, L. ivanovii, (Seeliger, H. P. R., J. Rocourt, A. Schrettenbrunner, P. A. D. Grimont and D. Jones. 1984. Listeria ivanovii sp. nov. Int. J. Syst. Bacteriol. 34:336-337), L. innocua (Seeliger, H. P. R. 1981. Nonpathogenic Listeriae: L. innocua sp. Zbl. Bact. Parasit. Infect. I Abt. Orig. A. 249:487-493), L. welshimeri and L. seeligeri (Rocourt, J. and P. A. D Grimont. 1983. Listeria welshimeri sp. nov. and Listeria seeligeri sp. nov. Int. J. Syst. Bacteriol. 33:866-869) as well as L. grayi (Rocourt, J., P. Boerlin, F. Grimant, C. Jaquet, and J. C. Piffaretti. 1992. Assignment of L. grayi and L. murrayi to a single species, L. grayi, with a revised description of L. grayi. Int. J. Syst. Bacteriol. 42:171-174).

Within the genus Listeria, only Listeria monocytogenes and Listeria ivanovii are opportunistic pathogens, with L. ivanovii being the infectious agent of bovine mastitis and sheep encephalitis and is not a human pathogen (Gray, M. L. and A. H. Killinger. 1966. Listeria monocytogenes and listeric infections. Bact. Rev. 30:309-382). Listeria monocytogenes is a human opportunistic pathogen.

Listeria are parasites which infect the host cell and can multiply therein (Kreft, J. 1992. Listeria

monocytogenes—ein Modell für fakultativ intrazelluläre Bakterien. BioEngineering 1:65-71). The first step of the infection is the adhesion to the cell membrane of the target cells, like macrophages or enterocytes. The bacteria enclosed in a phagosom are taken up by active phagocytosis which were induced by the Listeria, whereby the cell wall bound by the Listeria protein internalin is involved. Thus, it is possible for Listeria to overcome the immune system of the infected organism and even to break through the placental barrier (Hof, H. 1990. Listerien—eine Herausforderung für die Diagnostik. Immun. Infekt. 18:35-39). The inclusion of haemolysins listeriolysin O into the membrane of the phagosomes leads to the lysis of the phagosomes, whereby the Listeria are liberated into the cytoplasm and can multiply. The further advance into neighbouring cells results from polymerisation of host-actin. The risk of being infected by Listeria is increased for pregnant women, unborn and newborn babies, senior citizens and people who have weakened immune systems (Bloome, C. V. 1993. Listeriosis: can we prevent it? ASM News. 59:444-446). The infection is a systemic infection, which concerns mainly the central nervous system, the circulatory parts as well as the gastro-intestinal tract. Symptoms are inter alia meningitis, sepsis, endocarditis, gastroenteritis and lung infection.

During pregnancy, early contractions, abortions or still-born babies may occur. If the newly born are infected by Listeria, two clinical forms are known, which are early onset and late onset. With early onset Listeriosis, the child is already infected in utero and the actual disease occurs shortly after the birth. With late onset Listeriosis, the infection occurs probably during childbirth or in a Listeria-infected environment. The infection then becomes manifest one to several weeks after the birth. The lethality rate both for early and

late onset is up to 20% (Slutsker, L. and A. Schuchat. 1999. Listeriosis in humans. In: E. T. Ryser and E. H. Marth [Ed.]. Listeria, Listeriosis and Food Safety. Marcel Dekker. New York.).

According to their ubiquitous occurrence, the Listeria bacteria can be detected in raw food, in the earth as well as in the faeces of humans and animals. They are very undemanding and can survive under different environmental conditions longer than other non-spore forming bacteria. However, it is certain that they are killed under normal pasteurisation conditions (71-72° C., 15 seconds). Therefore only food is dangerous which is eaten raw and which has been stored for a long time at refrigerator temperatures or which has been secondarily contaminated after pasteurisation (Rocourt, J. 1994. Listeria monocytogenes: the state of the science. Dairy Food Environ. Sanit. 14:70-82 and Krämer, J. 1997. Lebensmittelmikrobiologie, 3. Aufl., Eugen Ulmer Verlag, Stuttgart.).

Different categories of food can be involved in listeriosis. By now it is known that especially Serovars ½a, b, c and 4b are the infectious agents of the more serious disease conditions. Table 1 shows some known epidemic outbreaks in the past.

TABLE 1
Epidemiological Outbreak of Listeriosis

| | | | Lethality | |
|---|---|---|---|---|
| Year | Place | Cases | Rate [%] | Food |
| 1994 | Switzerland | 57 | 32 | Camembert |
| 1994-95 | France | 33 | 25 | Camembert |
| 1994/95 | Sweden | 8 | 25 | Smoked |
| | | | | Fish |
| 1996 | Illinois, | 45 | ? | Chocolate |
| | USA | | | Milk |
| 1998-99 | USA | 50 | 34 | Sausages |
| | | | | and Milk |
| 1998/99 | Finland | 18 | 22 | Butter |
| 1999 | France | 25 | 7 | Pig's |
| | | | | tongue |
| 2000-01 | USA | 12 | 5 | Mexican |
| | | | miscarriages | style |
| | | | | cheese |

As mentioned above, for a long period of time, the classification of Listeria was difficult. This was specifically as in the samples to be examined, the number of Listeria was oftentimes low and the further bacteria would be quite dominant. However, for the safety of people and the reliability of quality, reliable and quick detection methods are absolutely necessary. Conventional methods like the IDF Standard (143A:1995) of the International Dairy Federation for Milk and Milk Products have the disadvantage that they need a long phase of enrichment. This time is necessary to suppress the other bacteria occurring in food so that the Listeria will have a growth advantage. An alternative method to separate Listeria from the further matrix of food and thereby enrich them is magnetic separation. Therein, with the help of magnetic beads, bacteria are selected. To these particles, ligands, like

antibodies and lectins can be bound which have an affinity to the target cells. The application in pure culture is already quite promising, however problems arise if Listeria are detected from mixed cultures or food. In 1948 Gray et al, (Gray, M. L., H. J. Stafseth, F. Thorp, L. B. Sholl and W. F. Riley. 1948. A new technique for isolating Listerellae from the bovine brain. J. Bact. 55:471-476) developed the so-called cold enrichment, whereby the psychotroph nature of Listeria was used. At 4° C. they have a growth advantage vis-à-vis other bacteria, whereby the Listeria, however also multiply at a lower rate at this temperature. Therefore, the result of the cold enrichment might be complete only after several weeks.

Another method was the identification by illumination with light according to Henry, (Henry, B. S. 1993. Dissociation in the genus Brucella. J. Inf. Dis. 52:374-402) and Gray (Gray, M. L., H. J. Stafseth and F. Thorp. 1950. The use of potassium tellurite, sodium azide, and acetic acid in a selective medium for the isolation of Listeria monocytogenes. J. Bact. 59:443-444). According to this method a light source is reflected in a 45° angle on the bottom of a petri-dish. The Listeria colonies are identifiable by their typical reflection image. By now, numerous cultural methods are available which enable the addition of different selective agents which allow the growth of Listeria only. The Standard Method of the IDF 143A:1995 comprises a three-step detection reaction, namely selective enrichment, cultivation on a selective medium and identification of suspicious colonies according to bio-chemical or haemolytic characteristics.

However, this method takes up to seven days.

Alternative methods are based on immunological and molecular biological basis. Immunological methods are based on the detection of Listeria-specific antigens by complex formation with antibodies. Detection with fluorescent antibodies was described in 1988 by Smith and Archer, (Smith, J. L. and D. L. Archer. 1988. Heat induced injury in Listeria monocytogenes. J. Ind. Microbiol. 3:105-110). Application in food was however less successful, as a cross-reaction with anti-serum occurred. Donnelly and Baigent, (Donnelly, C. W. and G. J. Baigent. 1986. Method for flow cytometric detection of Listeria monocytogenes in milk. Appl. Environ. Microbiol. 52:689-695) improved this detection by flow-cytometry, however false positive results occurred. The reason for these disadvantages was the use of polyclonal antibodies, which cross-reacted with other gram-positive bacteria.

Farber and Speirs, (Farber, J. M. and J. I. Speirs. 1987. Monoclonal antibodies directed against the flagellar antigens of Listeria species and their potential in EIA-based methods. J. Food Prot. 50:479-484) isolated monoclonal antibodies against flagellar antigens of the species Listeria. They carried out enzyme immunoassay whereby the bacteria were included onto a nitro-cellulose filter and were detected by monoclonal antibodies as well as a secondary antibody which was conjugated with a peroxidase. The disadvantage of this method was the lack of species specificity within the genus Listeria and the use of a filter (Cassiday, P. K., Brackett, R. E. 1989. Methods and media to isolate and enumerate Listeria monocytogenes: a review. J. Food Prot. 52:207-214).

In ELISA Kits (enzyme-linked immunosorbent assay) a specific primary antibody is coupled with a solid phase (96-well Microplate). After the binding of antigen and

the removal of non-bound antigen, the secondary enzyme-conjugated antibody and the enzyme-substrate are added. The reaction-products (light or colour complexes) are measured.

Further solid phases for the immobilisation of antibodies can be ferro-magnetic particles, as they are used in magnetic separation.

In molecular biology specific DNA sequences of the organism to be detected are used. These target sequences are the basis of different virulence factors, which are, for example, specific in L. monocytogenes. Klinger et al, (Klinger, J. D., Johnson, A., Croan, D., Flynn, P., Whippie, K., Kimball, M., Lawrie, J., Curiale, M. 1988. Comparative studies of nucleic acid hybridisation assay for Listeria in foods. J. Assoc. Off. Anal. Chem. 71:669-673) described in 1988 a method wherein a 16S rRNA-sequence is used for the detection of Listeria in food and environmental samples. With this method it was possible to detect Listeria within 2.5 days.

Good results are also possible with the polymerase chain reaction (PCR). Therein, the desired nucleic acid sequence is limited by specific primers and is amplified with a thermo-stable DNA-polymerase in an exponential manner. A disadvantage of the immunological and molecular biological methods is that no colonies exist and that the results cannot be verified with the help of other tests.

A completely different method for the detection of Listeria are bacteriophages. Loessner and Busse, (Loessner, M. J. and Busse, M. 1990. Bacteriophage typing of Listeria species. Appl. Environ. Microbiol. 56:1912-1918) developed a phage characterisation, whereby the differentiation of Listeria isolates is possible even on

the strain or serovar level. A method for the release of Listeria nucleic acid and proteins with phagelysin ply118 was described in 1995 also by Loessner et al, (Loessner, M. J., Schneider, S., Scherer, S. 1995. A new procedure for efficient recovery of DNA, RNA and proteins from Listeria cells by rapid lysis with a recombinant bacteriophage endolysin. Appl. Environ. Microbiol. 61:1150-1152). Thereby, it is possible to liberate DNA, RNA or cellular proteins in a quick and efficient manner. It was also possible to introduce reporter-genes, which code for easily detectable products, into the phage-genome. The recombinant A511::luxAB-Phage as constructed in 1996 from Loessner et al (Loessner, M. J., C. E. D. Rees, G. S. A. B. Stewart and S. Scherer. 1996b. Construction of luciferase reporter bacteriophage A511::luxAB for rapid and sensitive detection of viable Listeria cells. Appl. Environ. Microbiol. 62:1133-1140) carries the luciferase gene luxAB of Vibrio harveyi. The expression of luciferase can be measured in a luminometer.

The nomenclature of the bacteriophages results from their specific host bacteria. For example, Listeria bacteriophages are those bacteriophages which infect the genus Listeria.

Since 1945 more than three hundred phages from the species of Listeria have been described, (Loessner, M. J., I. B. Krause, T. Henle, S. Scherer. 1994. Structural proteins and DNA characteristics of 14 Listeria typing bacteriophages. J. Gen. Virol. 75:701-710). Two Listeria phages will be shown to be especially relevant in the context of the present invention. These are the temperent Listeria phages A500 and A118. Both belong to the Siphoviridae family and A500 infects Listeria monocytogenes serovar 4b as well as several strains of Listeria innocua, namely serovar 6a and 6b.

A500 can adsorb to the serovar specific sugar substituents teichoic acids (Wendlinger, G., M. J. Loessner and S. Scherer. 1996 Bacteriophage receptors on Listeria monocytogenes cells are the N-acetylglucosamine and rhamnose substituents of teichoic acids or the peptidoglycan itself. Microbiol. 142:985-992).

As mentioned above, it is also possible to separate Listeria from further bacteria and pathogens in food as well as from the food itself, and enrich them by magnetic separation. Due to the problem which was still present even with magnetic separation, namely when Listeria should be isolated from mixed cultures or from food, further improvements of the magnetic separation were desired. In 1996 Loessner et al, (Loessner, M. J., A. Schneider, S. Scherer. 1996a Modified Listeria Bacteriophage lysin genes (ply) allow efficient overexpression and one-step purification of biochemically active fusion proteins. Appl. Environ. Microbiol. 62 :3057-3060) isolated and modified the lysin gene of the Listeria bacteriophage A500. This gene codes for the L-alanyl-D-glutamic acid peptidase and is expressed at the end of the lytic cycle of the virus multiplication in the host cell to liberate the new bacteriophage. The proteins have next to an enzymatic active domain (EAD), a cell wall binding domain (CBD) which leads the enzyme to a substrate in the peptidoglycane of the bacterial cell wall (Loessner, M. J., K. Kramer, F. Ebel, S. Scherer. 2002. C-terminal domains of Listeria monocytogenes bacteriophage murein hydrolases determine specific recognition and high-affinity binding to bacterial cell wall carbohydrates. Mol. Microbiol. 44:335-349).

Here it was shown that the endolysins ply118 and ply500 share a unique enzymatic activity and specifically hydrolyse Listeria cells at the completion of virus multiplication in order to release progeny phages. The domain structure was elucidated and the function of their unrelated and unique C-terminal cell wall binding domain (CBD) was examined. It was shown that both domains were needed for lytic activity. Fusions of CBDs with green fluorescent protein (GFP) demonstrated that the C-terminal 140 amino acids of ply500 and the C-terminal 182 residuals of ply118 are necessary and sufficient to direct the murein hydrolases to the bacterial cell wall. CBD500 was able to target GFP to the surface of Listeria cells belonging to serovar groups 4, 5 and 6, resulting in an even staining of the entire cell surface. In contrast, the CBD118 hybrid bound to a ligand predominantly present at septal regions and cell pools, but only on cells of Serovar groups ½, 3 and 7.

From EP 99 952414.3 in the name of S. Scherer and M. J. Loessner, a method for the detection of targeted cells by the use of the CBDs is known.

However, even in view of that disclosure it was desired at the time to provide further methods which would allow the enrichment of the target cells, especially with regard to the detection of target cells in food where the target cells would be mixed in culture with several other bacteria and would need to be enriched so as to make it possible to reliably detect them without the presence of other bacteria interfering with said detection.

Moreover, there was a need for an improved and advantageous method which would allow the reliable detection and enrichment of target cells with the use of cell wall binding domains.

Furthermore, there was a need for improved biochips as well as a method of using them, wherein said biochips comprised CBDs.

The above objects as well as the preferred embodiments are achieved by the invention as described according to the independent claims as enclosed herewith. Further preferred embodiments are disclosed in the dependent claims.

It shall be understood that all references and citations mentioned herein shall be incorporated by said reference in their entirety.

The following figures are included with this application.

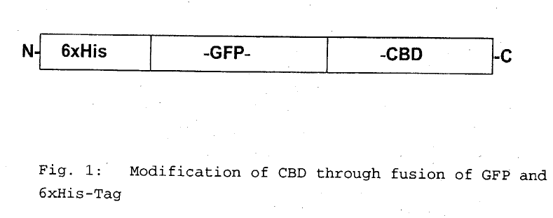

FIG. 1: Modification of CBD by fusion with GFP and 6×-HisTag.

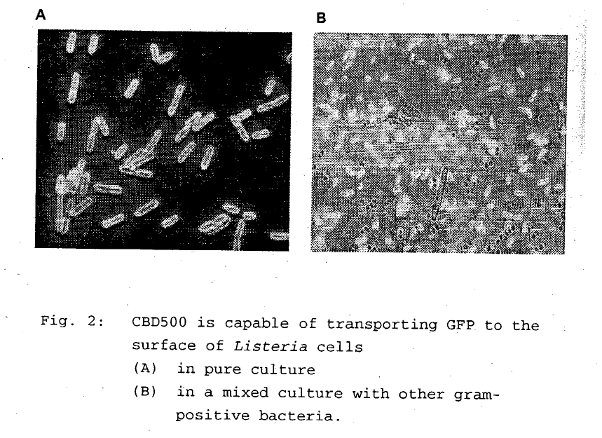

FIG. 2: HGFP-CBD500 on the surface of Listeria cells.

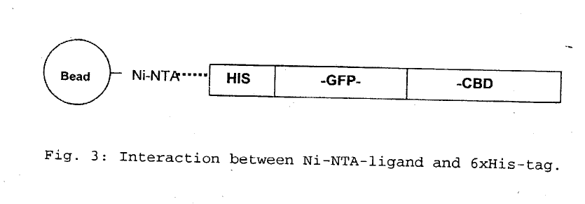

FIG. 3: Interaction between Ni—NTA ligands and 6×-HisTag

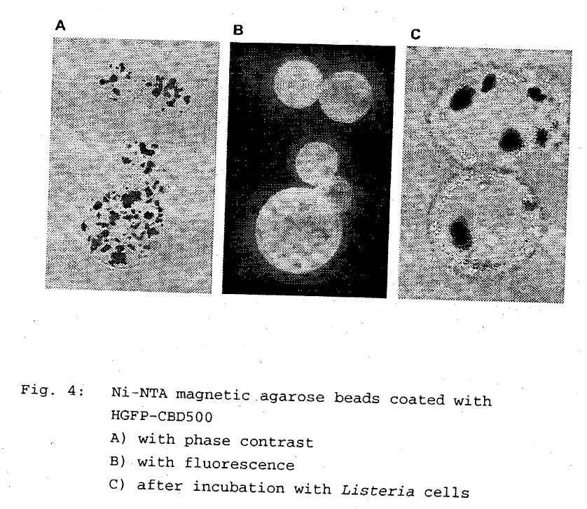

FIG. 4: Ni—NTA magnetic agarose beads, coated with HGFP-CBD500.

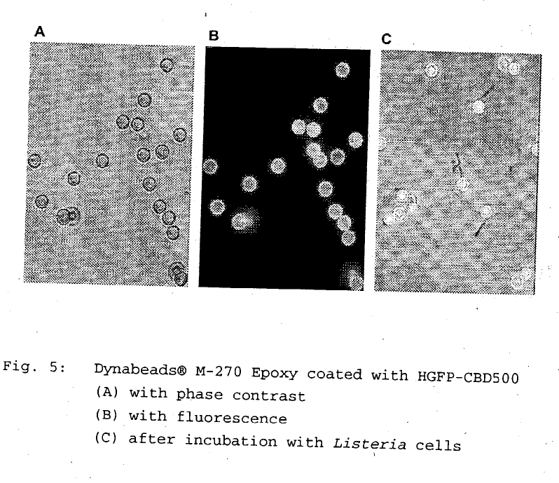

FIG. 5: Dynabeads® M-270 epoxy coated with HGFP-CBD 500.

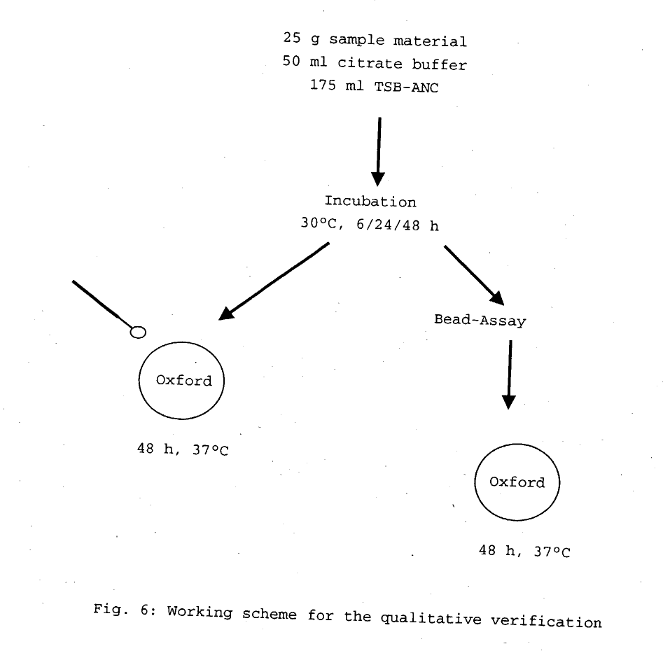

FIG. 6: Working graph for qualitative detection.

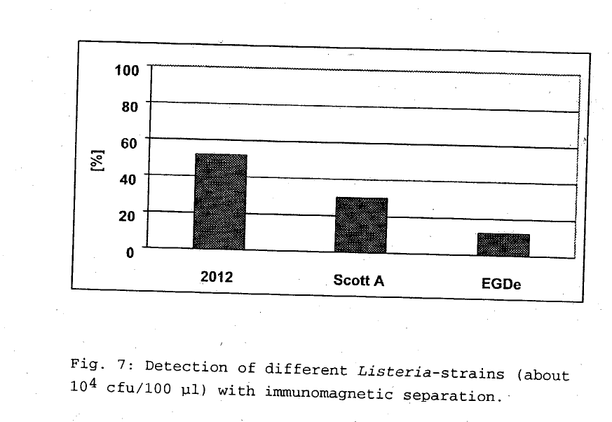

FIG. 7: Detection of different Listeria strains with immunomagnetic separation.

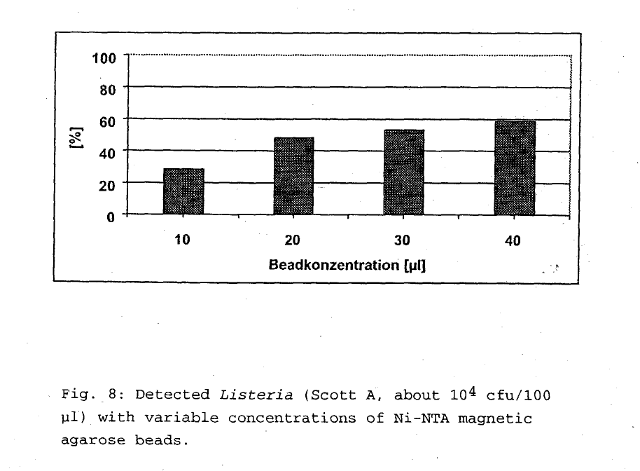

FIG. 8: Detected Listeria with variable Ni—NTA magnetic agarose bead concentrations.

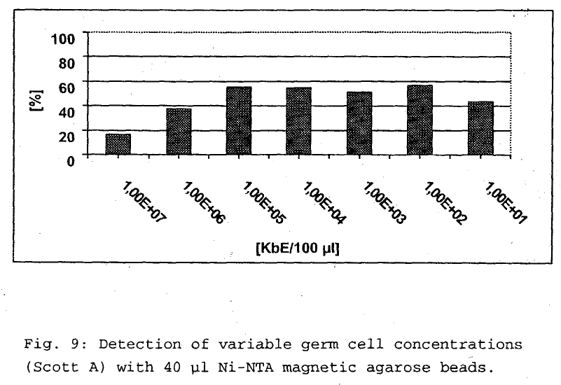

FIG. 9: Detection of different bacterial concentrations (Scott A) with 400 μl Ni—NTA magnetic agarose beads.

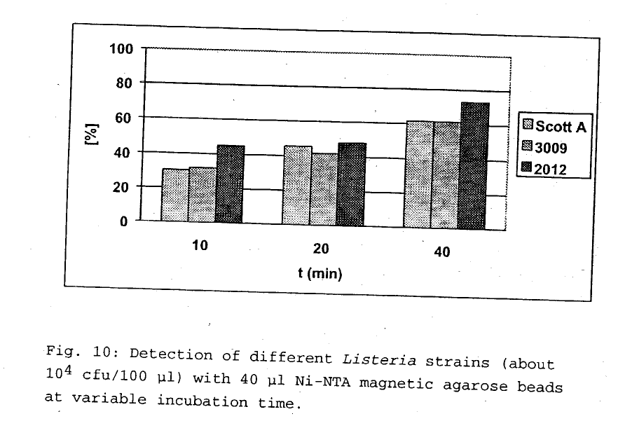

FIG. 10: Detection of different Listeria strains with 40 μl Ni—NTA magnetic agarose beads at variable incubation.

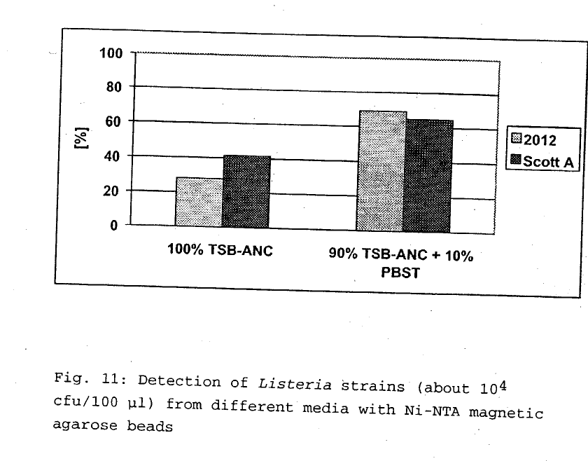

FIG. 11: Detection of Listeria strains from different media with NI—NTA magnetic agarose beads.

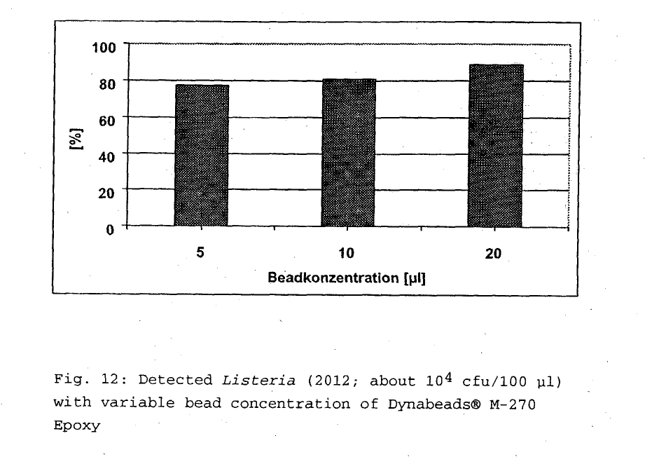

FIG. 12: Detected Listeria at variable bead concentration with Dynabeads® M-270 epoxy.

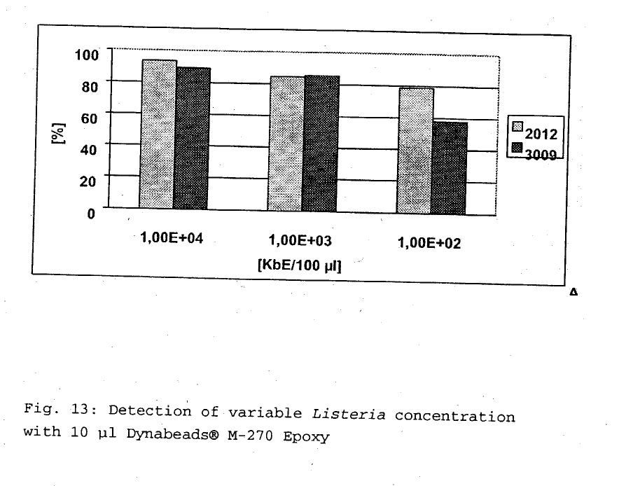

FIG. 13: Detection of variable Listeria concentrations with 10 μl Dynabeads® M-270 epoxy.

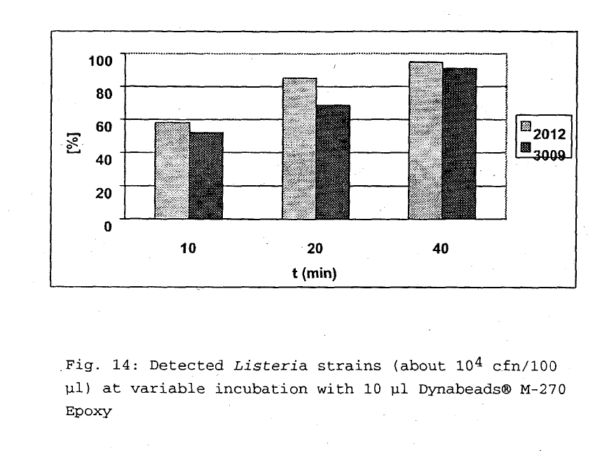

FIG. 14: Detected Listeria strains at variable incubation with 10 μl Dynabeads® M-270 epoxy.

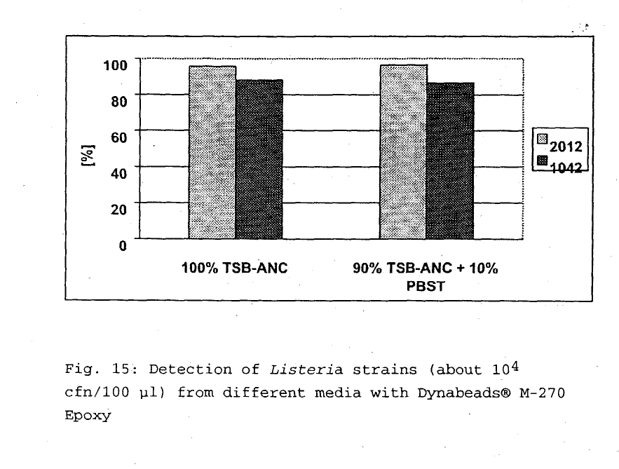

FIG. 15: Detection of Listeria strains from different media with Dynabeads® M-270 epoxy.

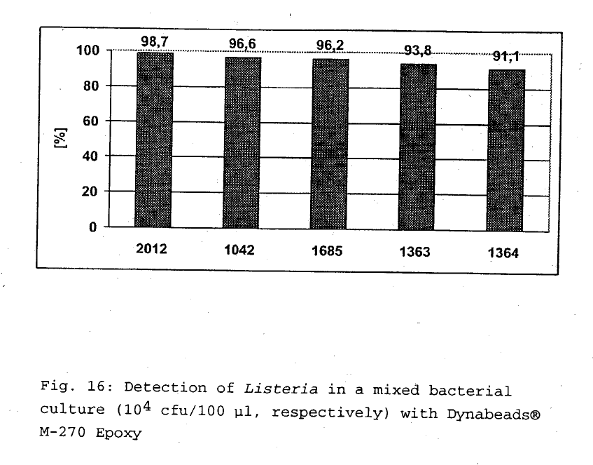

FIG. 16: Detection of Listeria from a bacterial mixed culture with Dynabeads® M-270 epoxy.

The invention is characterised by a method for the enrichment of target cells by binding wherein the method comprises the following steps:

selection of proteins which specifically bind the target cells, provision of the protein domains which are responsible for the binding to the cell wall (CBD) as protein fragments, wherein these protein fragments do not have any hydrolytic activity, binding of the CBDs to a solid phase, contacting the CBDs as obtained according to step (c) with a sample which comprises the target cells, and selective enrichment of said target cells.

“Target cells” as mentioned herein are defined as being all those cells which can be enriched by the method according to the present invention. Specifically, target cells are cells which shall be enriched or detected in a sample, e.g. a food sample or an environmental sample. Furthermore, target cells are specifically pathogens which might be encountered in food or other samples. According to a preferred embodiment, the target cells are selected from the group consisting of bacteria and bacterial spores, yeast, fungi and fungal spores, plant cells as well as animal cells. In a preferred embodiment, the target cells are selected from gram-positive bacteria, which include Aeromonas hydrophila, Bacillus anthracis, Bacillus cereus, Campylobacter jenuni, Clostridium botulinum, Clostridium perfringens, Clostrodium tyrobutyricum, Escherichia coli:H7 and other enteroxin-producing strains, Plesiomonas shigelloides, Salmonella species, Shigella species, Staphylococcus aureus, Streptococcus faecalis, Streptococcus pneumoniae, Streptococcus pyogenes, Vibrio cholerae, Vibrio parahaemolyticus, Vibrio vulnificus, Yersinia enterocolitica, Yersinia pseudotuberculosis. Most specifically, the target cells are selected from the group consisting of the genus Listeria, specifically, the human or animal pathogens within the genus Listeria, namely Listeria monocytogenes or Listeria ivanovii. Most specifically, the target cells according to the present invention belong to Listeria monocytogenes.

The term “CBD” i.e. “cell wall binding domain” is supposed to encompass herein all those protein domains which are part of proteins which specifically bind to the cell wall of the target cells. Antibodies will not be expressly included in said definition. According to a further preferred embodiment, the term “CBD” is also not supposed to encompass lectins. Furthermore, said CBDs are supposed to be those protein fragments which do not

have any hydrolytic activity. The cell wall binding domain is that part of the cell wall binding protein which is necessary and sufficient for the cell wall binding ability.

They are further preferably defined as being derived from hydrolytic enzymes of bacteriophage origin, which are capable of specific binding to bacteria. “Derived from” in this context refers to those CBDs which maintain their binding ability, but have no significant hydrolytic activity. No specific hydrolytic activity in this context is intended to describe the situation whereby the hydrolytic activity is not sufficient to prevent the CBDs' application to enrich and/or detect the bacteria hydrolysed.

Examples of CBDs are proteins or enzymes which selectively bind to the walls of cells. It is well known that such proteins usually have a domain structure, whereby part of the polypeptide chain in the native structure is able to recognise and bind specific molecules or molecular conformations on the surface of cells. Such molecules are, for example, the cell wall hydrolases as coded by bacteriophages, (Microbiol. Ref. 56, page 5430-5481. 1992); cell wall hydrolases of bacteria like, for example, lysostaphin (Mol. Gen. 209, page 563-569. 1987) and different autolysins. Further encompassed are recepter molecules coded by the DNA of bacteriophages and other viruses which are specific for yeast, fungi and eucaryotic cells, which combine to cell walls and also those cell wall proteins coded by the cell DNA which is non-covalently associated with a cell wall of target cells.

The gene sequences coding for the CBDs can be derived from the corresponding genetic information of the cells or viruses which code for the cell wall binding proteins.

In the following, some exemplary CBD's of Listeria bacteriophages are further described. The phage-lysin (Ply) or endolysins are L-Alanyl-D-glutamic acid peptidases which hydrolyze the peptidoglycan in bacterial cell walls. They belong to the so-called “late genes” during the lytic cycle of bacteriophages as they are produced at the end of gene expression in the lytic cycle of phage multiplication. The enzymes reach their substrate with the help of holin-proteins which destroy the cell membrane. Endolysins thereby enable

quick lysis of the host whereby the progeny of the bacteriophages can be liberated.

Ply 500 is formed by the Listeria bacteriophage A500 and consists of two functional domains. The N-terminal domain comprises the enzymatic active domain while the C-terminal domain comprises the cell wall binding domain, namely CBD 500. CBD is thereby necessary so as to direct the enzyme to the substrate and lends the enzyme its specificity.

CBD500 was modified in a preferred embodiment of the present invention by fusion with GFP, namely green fluorescent protein, and an N-terminal motive, consisting of 6 histidine residues which was defined as a “His-tag” (FIG. 1). The expression of the hybrid genes which code for HGFP-CBD500, was carried out in Escherichia coli. The purification of the proteins was carried out by Ni—NTA affinity chromatography.

It could be shown that both in pure as well as in mixed-culture, CBD 500 was able to lead GFP to the cell surface of Listeria of serovar 4, 5 and 6, whereby the cells were fluorescent green (FIGS. 2a and 2b).

The binding between CBD-proteins and the cell wall ligands is based on ionic interaction and is dependent on pH and NaCl content of the buffer. It could be confirmed that after 15 seconds the cell walls were already saturated with GFP.

The “solid phase” as mentioned in step (c) above, is meant to encompass any solid phase known to a person skilled in the art of molecular biology or biochemistry. Said solid phase can be a hydrophobic or hydrophilic surface, with a hydrophilic surface being preferred in the case of Listeria CBDs. The binding is carried out preferably by covalent binding. Most preferably, the solid phase consists of beads, which can be, according to the preferred embodiment, latex beads.

“Beads” in the context of the present application are meant to indicate essentially sphere shaped particles. However, all other forms are possible so that the beads according to the present invention are not limited to a sphere shape. Other shapes, like oval-shaped beads or rod-shaped beads are exemplary for further possible bead shapes. The beads according to the present invention are preferably latex beads, however, other compositions are possible as well, with polymers being preferred, specifically polystyrene or polyvinylalcohol. All other beads, known to a person skilled in the art of molecular biology or biochemistry are encompassed by said term as well.

According to the specifically preferred embodiments the latex beads according to the present invention have an average surface of between 10 and 1000 μm²/bead, preferably between 10 and 100 μm²/bead, especially preferred between 20 and 50 μm²/bead and an average diameter of 1 to 40 μm, preferably 1 to 10 μm, especially preferred 2 to 5 μm.

According to further preferred embodiments the latex beads are magnetic hydrophilic beads.

By the use of magnetic hydrophilic beads, which are, according to further preferred embodiment, pre-activated with hydrophilic epoxy groups, a magnetic separation of the target cells from other cells and further matrix material in e.g. a food or environmental sample can be carried out.

As mentioned above, target cells to be detected from food samples must usually be isolated from complex and dominant further cells and must for this means be enriched vis-à-vis said further cells and matrix material. A conventional detection method consists of selective enrichment, cultivation on a selective medium and further identification of suspicious colonies according to e.g. biochemical characteristics. However, the more recent methods which have their basis in immunology or molecular biology, also make further phases of enrichment necessary to detect very low numbers of target cells.

In many cases it is preferred to pre-enrich e.g. bacteria (i.e. target cells), in order to raise their numeber to more easily detectable levels.

A possibility to reduce the time necessary for enrichment is the specific magnetic separation of the target cells from a pre-enrichment culture medium. Thereby, not only the whole time required to carry out a test is shortened but also the sensitivity of the further detection method is improved. The cells are immobilised on ferromagnetic particles and can then be detected by usual methods. The necessary treatment equipment is—in addition to the magnetic beads mentioned above—an affinity ligand, which in the present case, is a CBD, as well as a magnet.

The magnetic particles usually used are mainly super-paramagnetic particles. Only in the presence of an external magnetic field do they have magnetic characteristics. With a magnet they can easily be separated from a suspension, however, without said magnetic field, they distribute homogenous in a solution. On their surface, different specific CBDs can be coupled to bind the target cells and separate them from the further suspension.

Application of immunomagnetic separation for isolation of Listeria monocytogenes, was described for the first time by Skjerve et al in 1990. (Skjerve, E., L. M.

Rorvik, O. Olsvik. 1990. Detection of Listeria monocytogenes in foods by immunomagnetic separation. Appl. Environ. Microbiol. 56 :3478-3481).

The detection limit from food was 2×10² cells per millilitres, (Uyttendaele, M., I. Van Hoorde, J. Debevere. 2000. The use of immnomagnetic separation as a tool in a sample preparation method for direct detection of Listeria moncytogenes in cheese. Int. J. Food Microbiol. 54:205-212) used inter alia IMS for the preparation of samples to detect Listeria monocytogenes without enrichment directly from cheese. Cell counts from 0.5 to 1.5 CFU per gram of cheese were detected. However, it could also be seen that the antibodies bind not only to the target cells. The percentage of non-specific binding was too high. Due to the high viscosity it was not possible to directly separate the target cells from the cheese by immunomagnetic separation. The binding of Listeria to the beads was possible only after dilution, centrifugation and enzymatic digest.

Duffy et al. detected Listeria spp in 1997 immuno-magnetically, (Duffy, G., J. J. Sheridan, H. Hofstra, D. A. MacDowell, I. S. Blair. 1997. A comparison of

immunomagnetic and surface adhesion immunofluorescent techniques for the rapid detection of Listeria monocytogenes and Listeria innocua in meat. Let. Appl. Microbiol. 24:445-450), to then make them visible by immunofluorescence. They could achieve the same results (approximately 10³ CFU per millilitre) as the standard methods in a shorter time (16 hours). However, they also reported non-specific binding. Other researchers encountered this problem as well.

By the use of the CBDs according to the present application, instead of the antibodies as used previously, it is possible to achieve a very specific binding with none of the disadvantages as mentioned above for the earlier magnetic separation methods. Therefore, rapid and reliable identification, detection as well as enrichment of Listeria and other target cells is possible via the use of CBDs.

Thereby, the surprising benefits of the CBDs include a higher specificity, high sensitivity and a more precise quantification; in addition they are clearly cheaper and quicker to use than the conventionally known methods.

Furthermore, it could be shown by the present inventors that especially covalent binding of the CBDs to a solid phase, wherein the solid phase consists of magnetic latex beads which are pre-activated with hydrophilic epoxy groups, wherein the latex beads have an average surface of between 10 and 1000 μm²/bead, preferably between 10 and 100 μm², especially preferred between 20 and 50 μm²/bead and an average diameter of 1 to 40 μm, preferably 1 to 10 μm, especially preferred 2 to 5 μm, result in very good and specific binding of the target cells with the CBD and very quick and reliable detection and enrichment of the desired target cells, as is also shown in the examples enclosed herewith.

The examples annexed here show the influence on the above parameters of the binding specificity of CBDs to Listeria species. This could be shown especially when the CBDs selected were CBD500 and/or CBD118.

CBD500 and CBD118 are those unrelated and unique C-terminal cell wall binding domains of the Listeria monocytogenes phage endolysins Ply118 and Ply500 as for example described in Molecular Microbiology 2002, (44 pp. 335 to 349 by M. J. Loessner et al).

They have proven to be specifically useful for rapid and reliable detection and amplification of bacteria of the genus Listeria, specifically, Listeria monocytogenes.

According to a preferred embodiment, the target proteins are selected from the group consisting of cell wall hydrolases coded by bacteriophages; bacterial cell wall hydrolases; autolysins; receptor molecules of bacteriophages and other viruses which are specific for yeast, fungi and/or eukaryotic cells; and cell wall proteins which are non-covalently associated with the cell wall.

Most preferably the proteins are selected from endolysins, bacteriophage lysins, lysins, murein-hydrolases and/or peptidoglykanhydrolases. According to a specific embodiment of the present application, the lysins are coded by bacteriophages for bacteria of the genus Listeria. Specifically, they are the above-mentioned endolysins Ply118 and Ply500 of Listeria monocytogenes phages. These belong to phages A118 and A500, respectively, which are members of the Siphoviridae family of double stranded DNA bacteria

viruses. Both phages absorb to serovar specific sugar substituents in the cell wall teichoic acids of their L. monocytogenes hosts. Both Ply118 and Ply500 possess unique catalytic activity: they cut the amide bonds between L-Ala and D-Gln within the peptide bridges cross-linking the Listeria Al γ-type peptidoglykan and where designated as L-alanyl-D-glutamate peptidases. The highly active enzymes exhibit stringent substrate specificity, i.e. they only lyse Listeria cells with very few exceptions among closely related bacteria. Ply118 and Ply500 exhibit sequence homology in the amino termini, apparently reflecting the identical enzymatic activity. In contrast, the two C-terminal domains show no significant sequence homology to each other, (Loessner M. J., G. Wendlinger, S. Scherer 1995. Heterogeneous endolysins in Listeria monocytogenes bacteriophages: a new class of enzymes and evidence for conserved holin genes within the siphoviralytic cassettes. Mol. Microbiol. 18:1231-1241), or to any protein from other organisms available from the current databases. It was shown by Loessner et al, 2002 (see above), that it was the C-terminal 140 amino acids of Ply500 and the C-terminal 182 residuals of Ply118 which were both necessary and sufficient to direct the murein-hydrolases to the bacterial cell wall, whereby, the C-terminal domain were designated CBD500 and CBD118, respectively.

According to further preferred embodiments, the cell wall binding polypeptide domains are derived from the nucleotide sequence of (a)gene, or the amino acid sequence of (a) gene product and are recovered therefrom.

Preferably, the gene products also comprise those gene products which are functional and effective only after a post-translational modification.

Gene sequences can then be combined according to means known to the person skilled in the art with new signals for the transcription and translation as well as replication structures like plasmid which allow the independent production of these protein fragments or polypeptides in heterologous organisms. The recombinant gene constructs, coding for novel proteins, are introduced into suitable organisms according to methods known to the person skilled in the art. Organisms which might be suitable in the present case are, for example Escherichia coli bacteria or Pichia pastoris yeasts. Thereby, recombinant products, proteins or polypeptides can be recovered. Subsequently, or even during the recombinant expression, the polypeptide chains can be

coupled with suitable particulate markers, amplification agents, dyes, isotopes, or marker genes like, for example fluorescent proteins. According to a preferred embodiment of the present invention, CBDs are thereby directly bound to a detectable marker, preferably by genetic translational fusion.

Said detectable marker may be a fluorescent protein, preferably GFP, which is green fluorescent protein, from Aequoria victoria, Science 263, page 802-805 (1994), especially preferred GFP mut-1, GFP mut-2 or GFP mut-3, which are mutated GFPs, which have been modified to provide an increased emission intensity, (Gene 173, page 33-38, (1996)). Also BFP, namely blue fluorescent protein, can be used. Numerous other fluorescent proteins are known in the art and could be used for the purpose of performing the invention, amongst others, these include red fluorescence protein, cyan FP, Yellow FP.

In an embodiment of the invention, different fluorescent proteins are used with fusions of more than one type of CBDs that can subsequently be used to perform

multiplexed detection of more than one distinct pathogenic or non-pathogenic bacteria in a sample. Such multiplex analysis can be performed in parallel or as a series of analysis.

According to a further preferred embodiment, the CBDs are directly bound with an amplifying substance which is detectable in further reactions, wherein the binding is preferably by genetic translational fusion. Amplifying substances are those usually used in the art and are known to persons skilled in the art. Most preferably, the amplifying substances are, for example, selected from biotin, peroxidase or phosphatase or another enzyme with a similar effect. The CBDs are preferably provided with detectable particulate markers, dyes, amplifying

substances or isotopes. Most preferably, the dye is a fluorescent dye. When the dye is a fluorescent dye, the amplifying substances are preferably biotin, peroxidase, phosphatase or another enzyme with a similar effect.

However, it is expressly pointed out that the primary CBD immobilized on the beads does not need to be labelled by GFP or similar fluorescent protein. Alternatively, the second CBD molecule, which is used as a detection marker for bound cells, could be fused to a fluorescent protein of a different nature, such as CFP, YFP, BFP, or dsRED, or to some other, non-protein fluorescent molecule. The highly sophisticated reader machines available today can easily differentiate between all these labels. All possible combinations and sequences of markers, detection molecules etc. known in the state of the art can, of course, be applied to the present technology as well. For example, the different embodiments of the present invention can be characterised in that the CBDs are directly bound to a detectable marker, preferably by genetic translational fusion. Furthermore, the different embodiments of the present invention can be characterised in that the target cells, immobilised by solid phase bound CBD are detected via a sandwich-CBD assay with detectable and/or modified secondary CBD molecules. Also in this case, the CBDs can be directly bound to a detectable marker, preferably by genetic translational fusion. In this embodiment; furthermore, the detectable marker can be a fluorescent protein, preferably GFP, BFP, especially preferred GFP mut-1, GFP-mut2 or GFP mut-3 or red fluorescent protein, cyan FP and yellow FP. Furthermore, in this embodiment it is possible that the CBDs are directly bound with an amplifying substance which is detectable in further reactions, wherein the binding is preferably by genetic translational fusion. Again, in that case, the amplifying substance can be biotin, peroxidase, phosphatase or another enzyme with a similar effect. Furthermore, in this embodiment, the CBDs can be provided with detectable particulate markers, dyes, amplifying substances or isotopes. The dye can be a fluorescent dye, the amplifying substance can be biotin, peroxidase, phosphatase or another enzyme with a similar effect. In this embodiment, the CBD can enable immobilization of the target cells to a solid surface by binding of the cell walls of the target cells, wherein said binding is carried out preferably at a pH between 7 and 10, more preferably at a pH between 8 and 9 and an NaCl content in the surrounding environment between 50 and 500 mM, preferably between 100 and 200 mM.

Further preferred, the CBDs enable immobilisation of the target cells to the solid surface by binding of the cell walls of the target cells. Said binding is most preferably carried out at a pH between 7 and 10, more preferably at a pH between 8 and 9, and an NaCl-content in the surrounding environment between 50 and 500 mM, preferably between 100 and 200 mM.

As mentioned above, the CBD polypeptides have specific characteristics which are in a way comparable to cell wall binding antibodies. They recognise specific epitopes, which can be proteins, carbohydrates, lipids or a combination of the same, on or in the cell wall and bind to these epitopes. The CBD polypeptides, however, have the advantage with regard to antibodies that they are very easy and cost effective to manufacture and that they are extremely specific, which is a further major difference between the CBD polypeptides and antibodies.

For the recognition of the C-terminal binding domain, namely the CBD within a structure, different methods are possible. For example, comparisons relating to the homology of the coding genes, namely nucleotide sequences, or the gene products, namely amino acid sequences or by independent expression of parts of the coding genes and cultures of recombinant bacteria and the subsequent determination of the C-terminal binding capability of the individual fragments of the original protein molecule are possible. However, it is important that every significant hydrolytic activity of the single fragments tested is not present in the CBD domain per se or is destroyed, e.g. by mutation.

Otherwise, if a significant hydrolytic activity remains, the CBD will not only bind to the cell wall but will also destroy it. Thereby, the effect of the present method would not be achievable. In a preferred embodiment, the CBDs have no significant hydrolytic activity.

According to a preferred embodiment, the CBDs used preferably are CBD 500 and/or CBD 118 as mentioned above. In the embodiment where the CBDs are preferably CBD 500 and/or CBD 118, the target cells are preferably cells of the species Listeria monocytogenes.

If CBD 500 is used, the target cells are preferably cells of the species Listeria monocytogenes serovar 4, 5 and or 6. When CBD 118 is used, the target cells are preferably cells of the species Listeria monocytogenes serovar ½, 3 and/or 7.

According to a further preferred embodiment, when the CBDs are CBD 118, the target cells are growing cells of the species Listeria monocytogenes.

According to a further preferred embodiment, the binding of the target cells occurs via cell wall associated teichoic acid.

The present invention is furthermore directed to the use of the method described above for the detection, diagnosis, immobilization or enrichment of cells.

Also encompassed is a reagent kit for a method as defined above, which comprises additionally to conventional detection means which are known to a person skilled in the art, one or more CBDs which are obtained as defined above and bound to the target cells as defined above.

Finally, the present invention is also directed to a biochip which comprises a CBD as defined above. Preferably, the biochip is a BIACore® or SELDI-biochip® as known to a person skilled in the art. Most preferably, the biochip comprises two or more different CBDs on defined locations. Thereby, by contacting the biochip comprising different CBDs on a defined location with a sample comprising different bacteria, the

detection and diagnosis of the bacteria in said sample is possible by simply recognizing the specific pattern of bound target cells per CBD on the defined location on the biochip which relates to the appropriate respective target cell definition.

In the following, the present invention shall further be described by way of examples. However, it shall be understood that the examples as enclosed herewith are not intended to delimit the present invention in any manner but shall simply be understood in an illustrative manner.

1. Material and Methods

1.1 Nutrient Media and Buffers

The following nutrient media and buffers were used in the experiments for the elaboration of the present invention. The media were stored at room temperature and the plates at 4° C., if not indicated differently.

BHI-Bouillon (Brain-Heart-Infusion)

Ready-to-use substrate (Merck, Darmstadt), consisting of:

27.5 g nutrient substrate (brain-heart-extract, peptone)

2.0 g D(+)glucose

5.0 g NaCl

2.5 g disodium hydrogen phosphate

pH: 7.4±0.2

dissolve 37.0 g ready-to-use substrate in 1000 ml distilled H₂O, autoclave for 15 min

Use: culture and replication of the Listeria strains

BHI-Agar

37.0 g ready-to-use substrate (see above)

14.0 g agar

dissolve in 1000 ml distilled H₂O, autoclave for 15 min, fill 12 ml, respectively, in petri dishes, store at 4° C.

Use: growth of Listeria

PC-Bouillon (Plate Count)

5.0 g casein-peptone

2.5 g yeast extract

1.0 g (D+)-glucose

pH: 7.0

dissolve in 1000 ml distilled H₂O, autoclave for 15 min

Use: culture and replication of Bacillus subtilis, Enterococcus faecalis, Staphylococcus aureus, Escherichia coli, Pseudomonas fluorescens

LB-Bouillon (Luria-Bertani)

15.0 g tryptone (casein-peptone, digested with trypsin)

8.0 g yeast extract

5.0 g NaCl

pH: 7.8

dissolve in 1000 ml distilled H₂O, autoclave 15 min

Use: culture and replication of E. coli JM 109 (HGFP-CBD500)

M17-Bouillon (According to Terzaghi; Oxoid)

ready-to-use substrate consisting of:

5.0 g soy bean peptone

2.5 g meat peptone

2.5 g casein-peptone

2.5 g yeast extract

5.0 g meat extract

5.0 g D(+)-lactose

0.5 g ascorbic acid

19.0 g Na-β-glycerophosphate

0.25 g magnesium sulfate

pH: 7.2±0.2

dissolve 42.5 g ready-to-use substrate in 1000 ml distilled H₂O, autoclave for 15 min

Use: culture and replication of Lactococcus garvieae

MRS-Bouillon (de Nan, Rogosa, Sharpe; Oxoid)

10.0 g peptone

8.0 g meat extract

4.0 g yeast extract

20.0 g glucose

1 ml Tween 80

2.0 g K₂PO₄

5.0 g sodium acetate×3 H₂O

2.0 g triammoniumcitrate

0.2 g magnesium sulfate×7 H₂O

0.05 g Manganese sul fate×H₂O

pH 6.2±0.2

dissolve 52.0 g ready-to-use substrate in 1000 ml distilled H₂O, autoclave for 15 min

Use: culture and replication of Lactobacillus brevis

TSB (Trypticase Soy Broth)

17.0 g casein-peptone

3.0 g NaCl

2.5 g K₂HPO₄

2.5 g D(+)-glucose

6.0 g yeast extract

pH: 7.3±0.2

dissolve in 1000 ml H₂O, fill 175 ml, respectively, in a bottle (Schott), autoclave for 15 min

Use: enrichment bouillon for Listeria

ANC

Acriflavine 225 mg dissolved in 100 ml distilled H₂O,

sterile filtration

nalidixic acid 450 mg dissolved in 10 ml sterile 0.05 N NaOH

cycloheximide 225 mg dissolved in 10 ml 40% ethanol

Add inhibitors directly before use to each 175 ml TSB:

1.0 ml acriflavine

0.2 ml nalidixic acid

0.5 ml cycloheximide

Use: selection agents for Listeria

Citrate Buffer

17.0 g tri-sodium citrate dihydrate

pH: 7.5

dissolve in 1000 ml distilled H₂O

autoclave for 20 min

Use: homogenization of foodstuff

Listeria-selection-supplement

200.0 mg cycloheximide

10.0 mg colistin sulfate

2.5 mg acriflavine

1.0 g cefotetane

5.0 mg fosfomycine

dissolve in 5 ml ethanol:distilled H₂O (1:1) and add aseptically to 500 ml sterile Oxford-agar cooled down to 50° C.

Oxford-Agar

ready-to-use substrate (oxoid) constisting of:

39.0 g columbia-agar-base

1.0 g esculine

0.5 g iron(III)-ammoniumcitrate

15.0 g lithium chloride

pH: 7.0±0.2

dissolve 55.5 g ready-to-use substrate and additional 3 g of agar in 1000 ml distilled water, autoclave for 15 min, cool down to 50° C. and add Listeria-selection-supplement aseptically, store the plates in the dark

Use: selection medium for Listeria

Buffer A

500 mM NaCl

50 mM di-sodium hydrogen phosphate

5 mM imidazole

pH: 8.0

Dissolve in 1000 ml MilliQ, after autoclaving add 0.1% Tween 20 to the warm solution

Use: Wash-buffer for Ni—NTA magnetic agarose beads

Buffer B

500 mM NaCl

50 mM di-sodium hydrogen phosphate

250 mM imidazole

pH: 8.0

dissolve in 1000 ml MilliQ, after autoclaving add 0.1% Tween 20 to the warm solution

Use: Detach His-tagged CBD molecules from Ni—NTA ligands

Buffer C

300 mM NaCl

50 mM di-sodium hydrogen phosphate

200 mM imidazole

pH: 8.0

dissolve in 1000 ml MilliQ, after autoclaving add 0.1% Tween 20 to the warm solution

Use: Detach CBD-molecules and cells from Ni—NTA agarose beads.

Buffer A/B

A:B=9:1

Use: Wash-buffer for Ni—NTA magnetic agarose beads

PBS

120 mM NaCl

50 mM di-sodium hydrogen phosphate

pH: 8.0 (if not differently indicated)

dissolve in 1000 ml MilliQ, autoclave for 20 min

Use: dilution buffer

PBS/BSA

PBS with 0.1% BSA

pH: 7.4

Use: Storage buffer for Dynabeads® M-270 Epoxy

PBST

PBS with 0.1% Tween 20

Use: dilution buffer

PBST (10-Fold)

10-fold concentration of PBST

Use: pH-adjustment

Dialysis Buffer

100 mM NaCl

50 mM sodium-dihydrogen phosphate

pH: 8.0

dissolve in 1000 ml MilliQ, after autoclaving add 0.005% Tween 20 to the still solution

Use: dialysis

Na Phosphate

100 mM sodium-dihydrogen phosphate

pH: 7.4

Use: Washing of the Dynabeads® M-270 Epoxy

Ammonium Sulfate

3 M ammonium sulfate

pH: 7.4

dissolve in 1000 ml Na-phosphate, sterile filtration

Use: coating of Dynabeads® M-270 Epoxy

Ampicillin

1 g Ampicillin

dissolve in 20 ml MilliQ, sterile filtration, store at −20° C.

Use: plasmid selection

IPTG (1-isopropyl-β-D-1-thiogalactopyranoside)

1 g IPTG

add MilliQ up to 10 ml, sterile filtration, store at −20° C.

Use: induction of protein production

1.2 Bacteria

All Listeria strains (Tab. 2) used for the practical work are a part of the Weihenstephan Listeria Collection (WSLC).

The remaining bacterial strains (table 2) are derived from the Weihenstephan Collection (WS).

TABLE 2
Overview over the used bacterial strains

| Species, strain | Serovar |
|---|---|
| L. monocytogenes WSLC 1685 (Scott A) | 4b |
| L. monocytogenes WSLC 1042 | 4b |
| L. monocytogenes EGDe | 1/2a |
| L. monocytogenes WSLC 1363 | 4b |
| L. monocytogenes WSLC 1364 | 4b |
| L. innocua WSLC 2012 | 6b |
| L. ivanovii WSLC 3009 | 5 |
| Bacillus subtilis 168 | — |
| Enterococcus faecalis WS 1761 | — |
| Staphylococcus aureus WS 2268 | — |
| Escherichia coli WS 1323 | — |
| Pseudomonas fluorescens WS 1760 | — |
| Lactobacillus brevis WS 1025 | — |
| Lactococcus garvieae WS 1029 | — |
| E. coli JM 109 (pHGFP-CBD500) | — |

The experiments were performed with cultures in the exponential growth phase. In the evening, 3 ml BHI medium were inoculated with Listeria. After an incubation (30° C.) overnight additional 7 ml fresh BHI were added to cause the bacteria to enter the growth phase again.

After centrifugation (7000 rpm, 10 min., 4° C., rotor 19777) and one wash-step with 10 ml PBS the cell pellet was resuspended in 5 ml PBS. The number of germs in the Listeria suspension was determined by means of the respective dilutions. Each 100 μl were plated out on a BHI-agar. The plates were incubated for 16 hours at 37° C. The numbers of germs in the overnight cultures for the food contaminations were determined by measuring their optical density.

1.3 Beads

Two different kinds of magnetic particles were used in the experiments, which were different in material, size and type of binding. They were tested for their capacity to serve as solid phases for the immobilization of HGFP-CBD500.

1.3.1 Dynabeads® Anti-Listeria

Dynabeads® anti-Listeria are latex beads (Dynal, Oslo, Norway) having polyclonal antibodies covalently bound to their surface. One aliquot of the enriched sample with the beads was incubated under continuous shaking at room temperature, thus resulting in a complex of the specific antibodies with Listeria. Afterwards the bead-bacteria-complex was separated from the suspension with a magnet, washed for 10 min in 500 ul PBST (pH 7.4) in a shaker (900 rpm, Elmi), resuspended in 100 μl PBST and plated out.

1.3.2 Ni—NTA Magnetic Agarose Beads

Ni—NTA magnetic agarose beads (Qiagen, Hilden) consist of agarose and contain magnetic particles. They have an average diameter of approx. 50 μm (20-70 μm) and an average surface of about 7800 μm²/bead. On their surface they have covalently bound nitrilotriacetic acid groups (NTA). These NTA groups complex divalent nickel ions.

The Ni—NTA-ligands adsorb the 6×His-Tag of HGFP-CBD500, in such a way that the C-terminal cell wall binding domain is always directed to the exterior (FIG. 3).

Between the Ni—NTA-ligands and the 6×His-Tag exists a reversible ionic bond, which makes it possible to detach the CBD molecules and the bound Listeria cells.

1.3.3 Dynabeads® M-270 Epoxy

Dynabeads® M-270 Epoxy have a constant diameter of 2.8 μm and a surface of 24.6 μm². They consist of highly crosslinked latex having magnetic particles embedded in its pores. The beads are surrounded by a hydrophilic layer of epoxy groups which allow for a covalent binding to proteins via primary amino groups. Thereby the arrangement of the CBD molecules cannot be controlled, since the bonds are can occur with all free amino groups. The CBD molecules with bound Listeria cells will not be detached, and the bead-bacteria-complex can be plated out.

1.4 Magnet

The magnet MPC®-S used for the practical work was obtained from Dynal Biotech. It is a permanent magnet with fittings for 6 Eppendorf tubes. With a magnet magnetic particles which are homogeneously distributed in a solution will be concentrated at the tube wall.

1.5 HGFP-CBD500

1.5.1 Isolation and Purification

First, an overnight culture of E. coli JM109 (pHGFP-CBD500) was established. To achieve this, 100 ml LB medium with an Ampicillin content of 100 μg/ml was inoculated and incubated in a shaker at 30° C. The next morning 10 ml of this overnight culture were added to 250 ml prewarmed (30° C.) LB-medium. The growth took place under continuous shaking until an OD₆₀₀-value of approx. 0.5 was reached. The protein production was induced by the addition of 1 mM IPTG. After additional 4 hours at 30° C. an incubation at 4° C. for 4 to 6 hours followed. After a centrifuging (7000 rpm, 10 min, 10° C., rotor JA 14) the cell pellet of 250 ml culture, respectively, was resuspended in 5 ml buffer A. Afterwards the samples were immediately frozen (−20° C.). After thawing the cell material was disrupted with a French press cell (SLM Aminco) at 100 MPa and centrifuged at 35000 rpm (rotor Ti 70) for 30 min. The supernatant was filtered aseptically (0.2 μm polyethersulfone membrane; Millipore) and frozen at −20° C.

The purification of the protein was performed by means of affinity chromatography in an FPLC-device (Pharmazia). In this step the material is bound to the matrix of the Ni—NTA-column. The imidazol ring, which is part of histidine molecules, has a very high affinity for the Ni²⁺-ions of the NTA-groups. By this means foreign proteins and other impurities can be washed out easily with 10% buffer B. Finally, the purified CBD may be detached from the column matrix with 100% buffer B.

After dialysis with two changes of buffer (4° C., 16 h) the sample material was concentrated to about 3 ml by means of an ultrafiltration unit (Fugisep-10, 10 kDa exclusion limited). For the determination of the protein content a protein assay (Nanoquant; Roth) was performed. The material was stored at −20° C. until use.

1.5.2 Coating of the Beads

1.5.2.1 Ni—NTA Magnetic Agarose Coated Beads

For the coating of the Ni—NTA magnetic agarose beads with HGFP-CBD500 no purified material was necessary. 200 μl CBD raw extract with a concentration of 2.3 mg/ml were incubated with 100 μl beads under continuous shaking (900 rpm, RT, 15 min, Elmi). For the removal of unbound CBD-material, two washes 500 μl buffer A and buffer A/B, respectively, were performed. This was done by carefully pipetting the suspension.

Successful coating can be monitored under the fluorescence microscope due to the fluorescence of the GFP (FIGS. 4A, B, C).

1.5.2.2 Dynabeads® M-270 Epoxy

For Dynabeads® M-270 Epoxy purified CBD was used, since foreign proteins may disturb the coating. The freeze-dried beads (60 mg) were first resuspended in 2 ml diglym (diethylene glycol dimethylether). Before taking out the desired amount of beads, it was necessary to mix the solution for 1-2 min to ensure a homogeneous distribution of the beads. 200 μl of beads were washed 2-times with 400 μl Na-phosphate for 10 min on a rotator (neolab) and then dissolved in 50 μl Na-phosphate, 50 μl purified CBD protein [2.5 mg/ml] and 100 μl ammonium sulfate. The coating was performed in a rotator at 4° C. for 16 hours and for additional 8 hours at room temperature. For the removal of the unbound CBD-material, the beads were washed 4-times with 400 μl PBS/BSA by carefully pipetting and then resuspended in 200 μl PBS/BSA. Again, the coating was also checked under the fluorescence microscope (FIG. 5).

1.6 Magnetic Separation

1.6.1 Anti-Listeria Dynabeads®

The strains WSLC 2012, Scott A and EGDe were tested. 100 μl of each culture (approx. 105 cfu/ml) were incubated with 4 μl Dynabeads® anti-Listeria and 96 μl PBST (pH 7.4) for 10 minutes at room temperature under continuous shaking (900 rpm, Elmi). Care has to be taken, that the beads did not settle during the incubation. The magnetic separation (MPC®-S) of the beads (3 min) allowed the removal of the suspension. The beads were washed in a shaker (900 rpm, RT, Elmi) with 500 μl PBST (pH 7.4) for 10 min, and were dissolved in 100 μl PBST. The respective dilutions were plated out on BHI-agar. The plates were incubated at 37° C. and analysed after 16 hours.

1.6.2 Ni—NTA Magnetic Agarose Beads

A total volume of 200 μl was used. Aliquots of a cell suspension were incubated with coated Ni—NTA beads at room temperature with continuous shaking (900 rpm, Elmi) to make the attachment of Listeria cells to the beads. The magnetic particles were separated from the suspension with the permanent magnet MPC®-S (Dynal Biotech, Oslo, Norway). The supernatant was removed, diluted in PBS and plated out on BHI-agar. The beads were washed with 500 μl PBST by careful pipetting to remove unbound cells. In some cases, the wash-fraction was also diluted and plated out. The detachment of the CBD molecules was performed by addition of 100 μl buffer C (600 rpm, 10 min, RT, Elmi). After a further separation of the magnetic beads with the magnet, the appropriate dilutions of the supernatant including the detached cells were plated out on BHI-agar.

To calculate the detection in percent one determination of the number of germs was performed in each case.

The plates were incubated at 37° C. for 16 hours.

1.6.2.1 Determination of Optimal Bead Concentrations

For the determination 10, 20, 30 and 40 μl beads were incubated for 30 min with 100 μl of a Listeria culture (Scott A, approx. 10⁵ cfu/ml).

1.6.2.2 Detection of Different Cell Concentrations

The bacterial culture (Scott A) in the log-phase was diluted in PBS to concentrations of about 10⁸, 10⁷, 10⁶, 10⁵, 10⁴, 10³, 10² cfu/ml. Of each dilution 100 μl were incubated for 30 min with 40 μl of beads.

1.6.2.3 Determination of the Optimal Incubation

The cultures of the strains WSLC Scott A, 2012 and 3009 were diluted in PBS to about 10⁵ cfu/ml. 100 μl thereof were incubated respectively for 10, 20 and 40 min with 40 μl beads.

1.6.2.4 Detection in Different Media

Cultures of the strains WSLC 1685 and 2012 were established in 100% TSB-ANC and 90% TSB-ANC+10% PBST

(10-fold), respectively. 100 μl of the bacterial cultures (about 10⁵ cfu/ml) were incubated with 40 μl of the beads for 40 min.

1.6.3 Dynabeads® M-270 Epoxy

In this case, again a total volume of 200 μl was used. 100 μl of a cell suspension were incubated with coated Dynabeads® at room temperature on a rotator (neolab). The beads were separated by means of a permanent magnet (Dynal Biotech, Oslo, Norway) (4 min). The supernatant was removed, appropriately diluted and plated out on BHI-agar. The beads were washed for 10 minutes at room temperature in 500 μl PBST on a rotator and then resuspended in 100 μl PBST. The appropriate dilutions of this suspension were plated out.

In order to calculate the percentage of detection, the number of germs was determined in each case.

The BHI-plates were incubated at 37° C., and analysed after 16 hours.

1.6.3.1 Determination of the Optimal Bead Concentration

For the determination 100 μl of a Listeria culture (WSLC 2012, about 10⁵ cfu/ml) were incubated with 5, 10 and 20 μl beads, respectively, for 30 min.

1.6.3.2 Detection of Different Cell Concentrations

Cultures of the strains WSLC 2012 and 3009, respectively, were diluted in PBS to concentrations of about 10⁵, 10⁴, 10³ cfu/ml. 100 μl of each dilution were incubated for 30 minutes with 10 μl beads.

1.6.3.3 Determination of the Optimal Incubation Time

100 μl each of the bacterial culture WSLC 2012 and 3009 (about 105 cfu/ml) were incubated for 10, 20 and 40 minutes with 10 μl beads.

1.6.3.4 Detection in Different Media

Cultures of the strains WSLC 1042 and 2012 were established in 100% TSB-ANC and in 90% TSB-ANC+10% PBST (10-fold) respectively. 100 μl of each bacterial culture (about 105 cfu/ml) were incubated for 40 min with 40 μl beads.

1.7 Isolation of a Mixed Bacterial Culture

For this experiment, mixed cultures of 7 different bacterial strains with one Listeria strain (WSLC 1042, 1363, 1364, Scott A and 2012), respectively were established. At first, overnight cultures of Bacillus subtilis, Pseudomonas fluorescens (PC, 30°), Enterococcus faecalis, Staphylococcus aureus, Escherichia coli (PC, 37° C.), Lactobacillus brevis (MRS, anaerob, 30° C.) and Lactococcus garvieae (M 17, anaerob, 37° C.) were established. The Listeria overnight cultures were established with the selection medium TSB-ANC. A mixed culture was established from the cultures which were diluted to about 10⁶ cfu/ml (1 Listeria strain, respectively). This 800 μl cell suspension was supplemented to 1000 ml with TSB-ANC+10% PBST (10-fold). 100 μl of the mixed culture were incubated for 40 min at room temperature with 10 μl beads on a rotator. After magnetic separation of the beads, the supernatant removed, diluted and plated out on Oxford-agar. The beads were resuspended in 100 μl PBST and also plated out on Oxford-agar. The plates were incubated for 48 hours at 37° C.

1.8 Detection in Artificially Contaminated Foodstuff

1.8.1 Foodstuff

For this experiment iceberg lettuce, ultra-pasteurized milk, turkey, minced meat, red spread cheese, camembert and smoked salmon were bought in local supermarkets and butcheries, respectively. With the exception of milk and salad, all foodstuff was first checked for the presence of Listeria according to the IDF standard method (IDF, 143A:1995). Each foodstuff sample (except milk and salad) was packed at 100 g in each case in sterile polyethylene bags and stored at −70° C. until use.

1.8.2 Contamination

The thawed foodstuff was contaminated with Listeria monocytogenes WSLC 1685 (Scott A). To this end, the 100 g portions in the polyethylene bags were artificially contaminated with 0.1, 1, 10, 10² cfu/g. The iceberg lettuce was chopped roughly, and in order to allow for an improved mixing 50 ml of PBS were added. The samples were stored for 2 days at 4° C.

1.8.3 Sample Preparation

After the storage, 25 g of foodstuff were taken out of each bag and homogenized in 50 ml citrate buffer in a Stomacher, and transferred into 175 ml TSB-ANC-Bouillon. This selection enrichment gives Listeria a growth advantage, since acriflavine suppresses Enterococcus and other gram-positive bacteria, nalidixic

acid suppresses gram-negative germs and cycloheximide suppresses yeasts and mildew.

An incubation at 30° C. was conducted, and after 6, 24 and 48 hours magnetic separations with Dynabeads® M-270 Epoxy were performed. To this end, 500 μl of the selective enrichment culture were removed. 50 μl PBST (10-fold) were added to adjust the pH-value. 110 μl sample, 10 μl Dynabeads® and 80 μl PBST (total volume=200 μl) were incubated on a rotator for 40 min at room temperature. After a magnetic separation, the beads were dissolved in 100 μl PBST, plated out on Oxford-agar and incubated for 48 hours at 37° C.

In parallel to each bead-assay, the IDF-standard method was performed: One loop (about 10 μl) of the selective enrichment was plated out on Oxford-agar and incubated for 48 hours at 37° C. (FIG. 6).

1.8.4 Analysis

The analysis of the plates was made after 24 and 48 hours.

Listeria are clearly visible after 48 hours as dark-brown or black colonies which are caved in the agar, and which have a dark spot in the middle. Due to their capability to digest esculin, the Listeria are surrounded by a brown halo.

2. Results

2.1 Dynabeads® Anti-Listeria

Dynabeads® anti-Listeria yielded very divergent detection rates. Whereas 52% of the WSLC 2012 strain was detected, only 30% of Scott A and 12.2% of EGDe were detected (FIG. 7). The observation with the microscope has shown that the antibody coupled beads have a tendency to agglutinate. Therefore, it is likely, that the number of colony forming units on the agar does not correspond to the cell number per se. Furthermore, it was observed that several cells have room enough on one bead. In this case, however, again the formation of only one colony forming unit occured.

2.2 Ni—NTA Magnetic Agarose Beads

2.2.1 Optimal Bead Concentration

As shown in FIG. 8, different bead concentrations lead to different detection rates of Listeria cells. If more beads and therefore more binding possibilities were present in the reaction volume, the detection rate was higher. By contrast the number of cells in the supernatant decreased with an increased bead concentration (Table 3). The best result with appox. 60% was obtained with 40 μl of beads. The number of beads in this optimal volume was microscopicly analysed by means of a Thoma counting chamber. Thereby, the surface of the optimal reaction volume can be calculated.

Surface Calculation for Ni—NTA Magnetic Agarose Beads

Radius r=25 μm

40 μl bead solution contain 172,000 beads.

O₁ Bead=4r²π=4(25 μm)²π=7,854 μm²/1 Bead

O₄₀ μl Beads=172,000*7,854 μm²=1,35×10⁹ μm² and 13.5 cm²/40 μl beads, respectively.

TABLE 3
Scott A and variable bead contractions

beads [μl]

| | 10 | 20 | 30 | 40 |
|---|---|---|---|---|
| supernatant [%] | 65.4 | 43.9 | 36.1 | 34.1 |
| detection [%] | 28.6 | 48.1 | 53.3 | 58.9 |

2.2.2 Detection of Varying Cell Concentrations

In this experiment, the detection of varying germ numbers was analysed. When using cell concentrations between 10⁵ and 10² cfu/100 μl, more than 50% of the cells were detected. With very high germ numbers the detection was low, since the binding capacity of the beads became insufficient. If the cell number decreases, the percentage of detected cells decreases also, because the distance between beads and cells increases (Table 4, FIG. 9).

TABLE 4
Ni-NTA magnetic agarose beads and variable numbers of germs

| | cfu/100 μl | | | | | | |
|---|---|---|---|---|---|---|---|
| | 10⁷ | 10⁶ | 10⁵ | 10⁴ | 10³ | 10² | 10¹ |
| supernatant | 75.5 | 50.8 | 32.8 | 30.2 | 38.5 | 31.7 | 49.8 |
| [%] | | | | | | | |
| detection [%] | 16.7 | 37.9 | 55.3 | 54.7 | 51.3 | 56.8 | 43.7 |

2.2.3 Optimal Incubation Time

As shown in the analysis, more cells were detected with increasing incubation time. It was found, that an incubation of 40 min is advantageous for all strains tested. The more Listeria have been immobilised on the beads, the less were detected in the supernatant (Table 5, 6, 7, FIG. 10).

TABLE 5
Scott A; variable incubation

| | min | | |
|---|---|---|---|
| | 10 | 20 | 40 |
| supernatant [%] | 56.5 | 44.2 | 22.9 |
| wash fraction [%] | 10.5 | 7.0 | 7.3 |
| detection [%] | 30.3 | 45.5 | 61.6 |

TABLE 6
3009; variable incubation

| | min | | |
|---|---|---|---|
| | 10 | 20 | 40 |
| supernatant [%] | 57.8 | 50.9 | 26.8 |
| wash fraction [%] | 8.3 | 3.5 | 2.0 |
| detection [%] | 31.7 | 41.8 | 61.5 |

TABLE 7
2012; variable incubation

| | min | | |
|---|---|---|---|
| | 10 | 20 | 40 |
| supernatant [%] | 46.7 | 41.2 | 7.8 |
| wash fraction [%] | 4.8 | 5.0 | 2.5 |
| detection [%] | 44.8 | 48.0 | 73.1 |

2.2.4 Detection in Different Media

Since the analyses of foodstuff were made in the selective medium TSB-ANC, the detection in 100% TSB-ANC and 90% TSB-ANC with 10% PBST (10-fold), respectively was compared. The pH-value of TSB-ANC is about 7.4, whereas the pH-value for the optimal binding of CBD to Listeria is 8.0. By addition of the 10-fold concentrated buffer, the detection level can be clearly increased. It was observed that the binding in pure TSB-ANC is weaker, since 20-30% of the cells were lost in the washing step.

TABLE 8
2012 in different media

medium

| | | 90% TSB-ANC + |
|---|---|---|
| | 100% TSB-ANC | 10% PBST |
| supernatant [%] | 37.4 | 14.0 |
| wash fraction [%] | 29.2 | 4.3 |
| detection [%] | 28.1 | 69.0 |

TABLE 9
Scott A in different media

medium

| | | 90% TSB-ANC + |
|---|---|---|
| | 100% TSB-ANC | 10% PBST |
| supernatant [%] | 36.5 | 27.1 |
| wash fraction [%] | 20.5 | 3.7 |
| detection [%] | 41.6 | 64.8 |

2.3 Dynabeads® M-270 Epoxy

2.3.1 Optimal Bead Concentration

Also for these beads the optimal bead concentration was determined first. Through an increase of the bead volume the detection rate could be enhanced. For future experiments, a concentration of 10 μl was chosen. Then the surface area with this reaction volume was calculated.

Surface calculation for Dynabeads® M-270 Epoxy radius r=1.4 μm 10 μl bead solution contain 2×10⁷ beads (Dynal, Oslo, Norway).

O₁ Bead=4r²π=4(1.4 μm)²π=24.6 μm²/1 beads

O₁₀ ul Beads=2×10⁷=24.6 μm²=4.92×10⁸ μm² and 4.92 cm²/10 μl beads, respectively

TABLE 10
2012 and variable bead concentrations

| | [μl] | | |
|---|---|---|---|
| | 5 | 10 | 20 |
| supernatant [%] | 10.0 | 8.2 | 2.4 |
| wash fraction [%] | 12.6 | 10.8 | 8.7 |
| detection [%] | 77.4 | 81.0 | 88.9 |

2.3.2 Detection of Varying Cell Concentrations

The following analyses show, that the detection of cells changes with variable numbers of germs. If less cells are present in the reaction volume, the distance between cells and beads increases and the detection rate decreases.

TABLE 11
Dynabeads ® M-270 Epoxy and
variable numbers of germs (2012)

| | cfu/100 μl | | |
|---|---|---|---|
| | 10⁴ | 10³ | 10² |
| supernatant [%] | 6.5 | 15.5 | 21.0 |
| detection [%] | 93.6 | 84.5 | 79.0 |

TABLE 12
Dynabeads ® M-270 Epoxy and
variable numbers of germs (3009)

| | cfu/100 μl | | |
|---|---|---|---|
| | 10⁴ | 10³ | 10² |
| supernatant [%] | 10.9 | 14.4 | 41.9 |
| detection [%] | 89.1 | 85.6 | 58.1 |

2.3.3 Optimal Incubation Time

This experiment shows, that again a longer incubation time improves the detection rate of Listeria with Dynabeads® M-270 Epoxy. In parallel, the cell number in the supernatant decreases. With the two analysed strains, an incubation of 40 min proved to be advantageous. Therefore, this incubation time was chosen for the next experiments.

TABLE 13
2012; variable incubation time

| | min | | |
|---|---|---|---|
| | 10 | 20 | 40 |
| supernatant [%] | 42.0 | 14.9 | 5.3 |
| detection [%] | 58.0 | 85.1 | 94.7 |

TABLE 14
3009; variable incubation time

| | min | | |
|---|---|---|---|
| | 10 | 20 | 40 |
| supernatant [%] | 47.9 | 31.3 | 9.0 |
| detection [%] | 52.1 | 68.7 | 91.0 |

2.3.4 Detection in Different Media

In this case it was checked again whether the selective enrichment bouillon for foodstuff analyses disturbs the binding of CBD and cells. As shown in the results, the addition of 10-fold PBST for the improvement of the detection rate was not absolutely necessary, since very good results were already obtained with pure TSB-ANC.

TABLE 15
2012 in different media

media

| | | 90% TSB-ANC + |
|---|---|---|
| | 100% TSB-ANC | 10% PBST |
| supernatant [%] | 4.4 | 3.6 |
| detection [%] | 95.6 | 96.4 |

TABLE 16
1042 in different media

media

| | | 90% TSB-ANC + |
|---|---|---|
| | 100% TSB-ANC | 10% PBST |
| supernatant [%] | 12.0 | 13.6 |
| detection [%] | 88.0 | 86.4 |

2.4 Comparison of the CBD-Coated Beads

For the assay with Ni—NTA magnetic agarose beads, 40 μl beads were necessary to obtain the highest detection rate of about 60% in pure cultures. With numbers of germs between 10² and 10⁵ cfu/100 μl the results were relatively constant. This optimal concentration contains about 172,000 beads and a total surface of about 13.5 cm² per assay. An increase of the detection rate to about 70% was obtained by a 40 min incubation time. The detection in different media has shown that the 10-fold concentrated PBST plays a role. The addition has almost doubled the detection rate.

With only 10 μl Dynabeads® M-270 Epoxy, more than 80% of the Listeria (ca. 10² bis 10⁵ cfu/100 μl) were separated from the pure culture. In this case again the incubation of 40 min proved to be advantageous. In this reaction volume 2×10⁷ beads are comprised, corresponding to a total surface of 4.9 cm² per assay. The addition of PBST (10-fold) in TSB-ANC has no marked effect, since the detection of Listeria also works very well in pure TSB-ANC. In contrast to the Ni—NTA magnetic agarose beads, it was found that Dynabeads® M-270 Epoxy were very well distributed during the incubation, whereby improved reproducibility of the results could be achieved. For this reason, the following experiments were only performed with Dynabeads® M-270 Epoxy.

TABLE 17
Comparison of the data with different beads

| | Ni-NTA Magnetic | Dynabeads ® M-270 |
|---|---|---|
| | Agarose Beads | Epoxy |
| volume/assay [μl] | 40 | 10 |
| number of | 172000 | 2 × 10⁷ |
| beads /assay | | |
| surface [μm²/bead] | ˜7800 | 24.6 |
| surface [cm²/assay] | 13.5 | 4.9 |

2.5 Detection in a Mixed Bacterial Culture

This experiment shows, how Listeria can be detected in the presence of other gram-positive and -negative bacteria. Therefore, the plating was carried out only on selective Oxford-agar, where only Listeria colonies with their characteristical morphology were seen after 48 hours. As shown in the analysis, more than 90% of the target cells were detected in each case.

TABLE 18
Detected Listeria strains in a mixed bacterial culture

| | Listeria strain | | | | |
|---|---|---|---|---|---|
| | 2012 | 1042 | 1685 | 1363 | 1364 |
| supernatant [%] | 1.3 | 3.4 | 3.8 | 6.2 | 8.9 |
| detection [%] | 98.7 | 96.6 | 96.2 | 93.8 | 91.1 |

2.6 Detection in Artificially Contaminated Foodstuff

The foodstuff was contaminated with different germ numbers, stored and analysed after 6, 24 and 48 hours with the IDF-method on the one hand, and with the bead-assay for Listeria on the other hand. Important criteria in this comparison are the detection limit and the possible advantage in time.

The results highlighted in grey in the tables demonstrate where the bead-assay was improved as compared with the IDF-standard method. The results of both methods were read on Oxford-agar-plates.

The meaning of the symbols is as follows:

| | | |
|---|---|---|
| [+] | 1-10 | cfu/plate |
| [++] | 11-50 | cfu/plate |
| [+++] | >50 | cfu/plate |

In most cases a shortened enrichment time of 24 hours was sufficient to detect the original contamination with the bead assay. After 24 and 48 hours there was often no significant difference between the standard method and the bead assay. However, it was sometimes possible to detect Listeria by magnetic separation after already 6 hours.

Iceberg Lettuce

TABLE 19
Detection in Iceberg lettuce

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | ++ | +++ | +++ | +++ |
| 1 | − | − | +++ | +++ | +++ | +++ |
| 10 | − | + | +++ | +++ | +++ | +++ |
| 100 | − | + | +++ | +++ | +++ | +++ |

In salad a contamination of 10 cfu/g was detected after only 6 hours with the bead-assay. With the IDF-method no detection was possible at this time. After 24 hours there was almost no difference between the methods.

Camembert

TABLE 20
Detection in Camembert

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | +++ | +++ | +++ | +++ |
| 1 | − | − | +++ | +++ | +++ | +++ |
| 10 | − | − | +++ | +++ | +++ | +++ |
| 100 | − | + | +++ | +++ | +++ | +++ |

Red Spread Cheese

TABLE 21
Detection in Red Spread Cheese

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | ++ | +++ | +++ | +++ |
| 1 | − | − | +++ | +++ | +++ | +++ |
| 10 | − | − | +++ | +++ | +++ | +++ |
| 100 | − | + | +++ | +++ | +++ | +++ |

Listeria were detectable both in Camembert as well as in Red Spread Chesse with an original contamination of 0.1 cfu/g after 24 hours and with both methods. With the bead-assay an original contamination of 100 cfu/g was detectable after 6 hours.

Smoked salmon

TABLE 22
Detection in smoked salmon

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | − | + | + | ++ |
| 1 | − | − | + | + | ++ | +++ |
| 10 | − | + | +++ | +++ | +++ | +++ |
| 100 | + | ++ | +++ | +++ | +++ | +++ |

In smoked salmon 10 cfu/g were detected with the bead-assay after 6 hours, whereas only 100 cfu/g could be detected with the IDF-method. After 24 hours the detection of 0.1 cfu/g was still not possible with the IDF-method.

Minced Meat

TABLE 23
Detection in minced meat

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | − | − | − | − |
| 1 | − | − | − | + | ++ | +++ |
| 10 | − | − | − | ++ | +++ | +++ |
| 100 | + | ++ | ++ | +++ | +++ | +++ |

A germ number of Listeria of about 100 cfu/g was detectable in minced meat with both methods after 6 hours. The detection limit by magnetic separation was

about 1 cfu/g after 24 hours, whereas it was about 100 cfu/g with the standard method. It was not possible to detect the original contamination of 0.1 cfu/g at any time point.

Turkey Cutlets

TABLE 24
Detection in turkey cutlets

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | − | + | + | ++ |
| 1 | − | − | − | + | + | ++ |
| 10 | − | − | + | ++ | ++ | +++ |
| 100 | − | + | ++ | +++ | +++ | +++ |

In turkey cutlets Listeria with a number of germs of about 100 cfu/g were detected after an enrichment of 6 hours. After 24 hours the bead-assay was better than the IDF-method at all levels of contamination.

Ultra-Pasteurized Milk

TABLE 25
Detection in ultra-pasteurized milk

| Original | 6 h | | 24 h | | 48 h | |
|---|---|---|---|---|---|---|
| Contamination | | Bead- | | Bead- | | Bead- |
| [cfu/g] | IDF | Assay | IDF | Assay | IDF | Assay |
| 0.1 | − | − | − | + | − | + |
| 1 | − | − | + | + | − | + |
| 10 | + | + | + | +++ | + | ++ |
| 100 | + | +++ | +++ | +++ | +++ | +++ |

enrichment: a number of germs of about 10 cfu/g was detectable. The detection of the original contamination of 0.1 cfu/g was possible after only 24 and 48 hours with the bead-assay.

Thereby, the CBSs are a highly effective tool for immobilization, enrichment and detection of Listeria as well as other pathogens or cells from e.g. food or environmental samples, especially in those cases where the target cells are in a low number compared to other cells or material present in the sample.

Especially effective is the combination of different CBDs, e.g. CBD 118 and CBD 500 to detect different bacteria or, in this case, detect all possible serovars of L-monocytogenes. A combination of different CBDs on a biochip would be particularly useful.

Furthermore, it can be contemplated to enrich and detect the cells immobilized on beads, e.g. by

plating or other cultivating methods, but also with more rapid methods like, e.g. PCR or with the help of the luciferase reporter bacteriophage A511::lux AB (see above) or by

the use of CBDs or antibodies labeled or bound with a fluorescent protein or dye or with an amplifying substance so that the immobilized cells can be detected by fluorescence, luminescence, color formation or other suitable method of detection. The CBD-related magnetic separation as described herein could then also effectively separate bacterial cells from PCR-inhibitors as comprised in food samples.

A person skilled in the art will understand that the foregoing description is meant to encompass all those modifications within the skill of a skilled person which fall within the spirit and scope of claims as enclosed herewith.

1. Method for the enrichment of target cells by binding wherein the method comprises the following steps:
(a) selection of proteins which specifically bind the target cells,
(b) provision of the protein domains which are responsible for the binding to the cell wall (CBD) as protein fragments, wherein these protein fragments do not have any hydrolytic activity,
(c) binding of the CBDs to a solid phase,
(d) contacting the CBDs as obtained according to step (c) with a sample which comprises the target cells,
(e) selective enrichment of said target cells, and
(f) optionally growing the target cells before or concurrently with steps (c), (d) and/or (e).

2. Method according to claim 1, wherein said binding in step (c) is a covalent binding.

3. Method according to claim 1, wherein said binding in step (c) is an immobilisation on a hydrophilic surface.

4. Method according to claim 2, wherein said solid phase consists of beads, preferably latex beads.

5. Method according to claim 4 wherein the latex beads have an average surface of between 10 and 1000 μm²/bead, preferably between 10 and 100 μm²/bead, especially preferred between 20 and 50 μm²/bead and an average diameter of 1 to 40 μm, preferably 1 to 10 μm, especially preferred 2 to 5 μm.

6. Method according to claim 5, wherein the latex beads are magnetic hydrophilic beads.

7. Method according to claim 6, wherein the magnetic hydrophilic beads are pre-activated with hydrophilic epoxy groups.

8. Method according to claim 5, whereby the proteins specifically binding to a target cell are selected from the following group:
Cell wall hydrolases coded by bacteriophages; bacterial cell wall hydrolases; autolysins; receptor molecules of bacteriophages and other viruses which are specific for yeast, fungi and/or eukaryotic cells; and cell wall proteins which are non-covalently associated with the cell wall.

9. Method according to claim 8, characterized in that the proteins are selected from endolysins, bacteriophage-lysins, lysins, murein-hydrolases and/or peptidoglykan-hydrolases.

10. Method according to claim 9, characterized in that the lysins are coded by bacteriophages for bacteria of the genus Listeria.

11. Method according to claim 5, characterized in that the target cells are selected from the group consisting of bacteria and bacterial spores, yeasts, fungi and fungal spores, plant cells and animal cells.

12. Method according to claim 5, characterized in that the cell wall binding polypeptide domains (CBD) are derived from the nucleotide sequence of (a) gene(s) and/or the amino acid sequence of (a) gene product(s) and are recovered therefrom.

13. Method according to claim 5, characterized in that the gene product(s) also comprise those gene products which are functional and effective only after post-translational modification.

14. Method according to claim 5, characterized in that the CBDs are directly bound to a detectable marker, preferably by genetic translational fusion.

15. Method according to claim 14, characterized in that the detectable marker is a fluorescent protein, preferably GFP, BFP, especially preferred GFP mut-1, GFP mut-2 or GFP mut-3, red fluorescence protein, cyan FP, Yellow FP.

16. Method according to claim 5, characterized in that the CBDs are directly bound with an amplifying substance which is detectable in further reactions, wherein the binding is preferably by genetic translational fusion.

17. Method according to claim 16, characterized in that the amplifying substance is biotin, peroxidase or phosphatase or another enzyme with a similar effect.

18. Method according to claim 5, characterized in that the CBDs are provided with detectable particulate markers, dyes, amplifying substances or isotopes.

19. Method according to claim 18 wherein the dye is a fluorescent dye.

20. Method according to claim 18 characterized in that the amplifying substance is biotin, peroxidase, phosphatase or another enzyme with a similar effect.

21. Method according to claim 5 wherein the CBD enable immobilization of the target cells to a solid surface by binding of the cell walls of the target cells and wherein said binding is carried out preferably at a pH between 7 and 10, more preferably a pH between 8 and 9 and an NaCl-content in the surrounding environment between 50 and 500 mM, preferably between 100 and 200 mM.

22. Method according to claim 5, characterized in that the target cells, immobilized by solid phase bound CBD are detected via a sandwich CBD assay with detectable and/or modified secondary CBD molecules.

23. Method according to claim 5, characterized in that the target cells immobilized by solid-phase bound CBD are detected via a sandwich-CBD-ELISA assay with detectable and/or modified secondary antibodies of the group of the immunoglobulins.

24. Method according to claim 5, characterized in that the target cells immobilized by solid phase bound primary antibodies of the group of immunoglobulins are detected via a sandwich-IG-CBD assay with detectable and/or modified secondary CBD molecules.

25. Method according to claim 5, characterized in that CBD, bound to a mobile solid phase, can bind target cells from a diluted and/or heterogenous mixture of cells and whereby in further steps the enrichment, isolation, purification and/or detection of said target cells is carried out.

26. Method according to claim 25, wherein the mobile phase consists of the magnetic beads as defined in claim 6.

27. Method according to claim 5, wherein the CBDs are CBD 500 and/or CBD 118.

28. Method according to claim 27, wherein the CBDs are CBD 500 and whereby the target cells are cells of the species Listeria monocytogenes Serovar 4, 5 and/or 6.

29. Method according to claim 27, whereby the CBDs are CBD 118 and whereby the target cells are cells of the species Listeria monocytogenes Serovar ½, 3 and/or 7.

30. Method according to claim 27, whereby the CBDs are CBD 118 and whereby the target cells are growing cells of the species Listeria monocytogenes.

31. Method according to claim 27, whereby the binding of the target cells occurs via cell wall associated teichoic acids.

32. Method for the specific recognition of target cells by binding wherein the method comprises the following steps:
a) selection of proteins which specifically bind the target cells;
b) provision of the protein domains which are responsible for the binding to the cell wall (CBD) as protein fragments, wherein these protein fragments do not have any hydrolytic activity;
c) covalent binding of the CBDs to a solid phase wherein the solid phase consists of beads, preferably latex beads,
d) contacting the CBDs as obtained according to step (c) with the sample to be examined, which comprises the target cells, and
e) optionally growing the target cells before or concurrently with steps (c), and/or (d).

33. Method according to claim 32 wherein the latex beads have an average surface of between 10 and 1000 μm²/bead, preferably between 10 and 100 μm²/bead, especially preferred between 20 and 50 μm²/bead and an average diameter of 1 to 40 μm, preferably 1 to 10 μm, especially preferred 2 to 5 μm.

34. Method according to claim 33, wherein the latex beads are magnetic hydrophilic beads.

35. Method according to claim 34, wherein the magnetic hydrophilic beads are pre-activated with hydrophilic epoxy groups.

36. Method according to claim 33, whereby the proteins specifically binding to a target cell are selected from the following group:
Cell wall hydrolases coded by bacteriophages; bacterial cell wall hydrolases; autolysins; receptor molecules of bacteriophages and other viruses which are specific for yeast, fungi and/or eukaryotic cells; and cell wall proteins which are non-covalently associated with the cell wall.

37. Method according to claim 36, characterized in that the proteins are selected from endolysins, bacteriophage-lysins, lysins, murein-hydrolases and/or peptidoglykan-hydrolases.

38. Method according to claim 37, characterized in that the lysins are coded by bacteriophages for bacteria of the genus Listeria.

39. Method according to claim 33, characterized in that the target cells are selected from the group consisting of bacteria and bacterial spores, yeasts, fungi and fungal spores, plant cells and animal cells.

40. Method according to claim 33, characterized in that the cell wall binding polypeptide domains (CBD) are derived from the nucleotide sequence of (a) gene(s) and/or the amino acid sequence of (a) gene product(s) and are recovered therefrom.

41. Method according to claim 33, characterized in that the gene product(s) also comprise those gene products which are functional and effective only after post-translational modification.

42. Method according to claim 33, characterized in that the CBDs are directly bound to a detectable marker, preferably by genetic translational fusion.

43. Method according to claim 42, characterized in that the detectable marker is a fluorescent protein, preferably GFP, BFP, especially preferred GFP mut-1, GFP mut-2 or GFP mut-3, red fluorescence protein, cyan FP, Yellow FP.

44. Method according to claim 33, characterized in that the CBDs are directly bound with an amplifying substance which is detectable in further reactions, wherein the binding is preferably by genetic translational fusion.

45. Method according to claim 44, characterized in that the amplifying substance is biotin, peroxidase or phosphatase or another enzyme with a similar effect.

46. Method according to claim 33, characterized in that the CBDs are provided with detectable particulate markers, dyes, amplifying substances or isotopes.

47. Method according to claim 46 wherein the dye is a fluorescent dye.

48. Method according to claim 46 characterized in that the amplifying substance is biotin, peroxidase, phosphatase or another enzyme with a similar effect.

49. Method according to claim 33 wherein the CBD enable immobilization of the target cells to a solid surface by binding of the cell walls of the target cells and wherein said binding is carried out preferably at a pH between 7 and 10, more preferably a pH between 8 and 9 and an NaCl-content in the surrounding environment between 50 and 500 mM, preferably between 100 and 200 mM.

50. Method according to claim 33, characterized in that the target cells, immobilized by solid phase bound CBD are detected via a sandwich CBD assay with detectable and/or modified secondary CBD molecules.

51. Method according to claim 33, characterized in that the target cells immobilized by solid-phase bound CBD are detected via a sandwich-CBD-ELISA assay with detectable and/or modified secondary antibodies of the group of the immunoglobulins.

52. Method according to claim 33, characterized in that the target cells immobilized by solid phase bound primary antibodies of the group of immunoglobulins are detected via a sandwich-IG-CBD assay with detectable and/or modified secondary CBD molecules.

53. Method according to claim 33, characterized in that CBD, bound to a mobile solid phase, can bind target cells from a diluted and/or heterogenous mixture of cells and whereby in further steps the enrichment, isolation, purification and/or detection of said target cells is carried out.

54. Method according to claim 53, wherein the mobile phase consists of the magnetic beads as defined in claim 34.

55. Method according to claim 33, wherein the CBDs are CBD 500 and/or CBD 118.

56. Method according to claim 55, wherein the CBDs are CBD 500 and whereby the target cells are cells of the species Listeria monocytogenes Serovar 4, 5 and/or 6.

57. Method according to claim 55, whereby the CBDs are CBD 118 and whereby the target cells are cells of the species Listeria monocytogenes Serovar ½, 3 and/or 7.

58. Method according to claim 55, whereby the CBDs are CBD 118 and whereby the target cells are growing cells of the species Listeria monocytogenes.

59. Method according to claim 55, whereby the binding of the target cells occurs via cell wall associated teichoic acids.

60. Method for the specific recognition of target cells by binding wherein the method comprises the following steps:
a) selection of proteins which specifically bind the target cells;
b) provision of the protein domains which are responsible for the binding to the cell wall (CBD) as protein fragments, wherein these protein fragments do not have any hydrolytic activity;
c) covalent binding of the CBDs to a solid phase wherein the solid phase consists of magnetic latex beads which are preactivated with hydrophilic epoxy groups wherein the latex beads have an average surface of between 10 and 1000 μm²/bead, preferably between 10 and 100 μm²/bead, especially preferred between 20 and 50 μm²/bead and an average diameter of 1 to 40 μm, preferably 1 to 10 μm, especially preferred 2 to 5 μm,
d) contacting the CBDs as obtained according to step (c) with the sample to be examined, which comprises the target cells, and
e) optionally growing the target cells before or concurrently with steps (c), and/or (d).

61. Method according to claim 60, wherein the proteins specifically binding to a target cell are selected from the following group:
Cell wall hydrolases coded by bacteriophages; bacterial cell wall hydrolases; autolysins; receptor molecules of bacteriophages and other viruses which are specific for yeast, fungi and eukaryotic cells; and cell wall proteins which are non-covalently associated with the cell wall.

62. Method according to claim 61, characterized in that the proteins are selected from endolysins, bacteriophage-lysins, lysins, murein-hydrolases and/or peptidoglykan-hydrolases.

63. Method according to claim 62, characterized in that the lysins are coded by bacteriophages for bacteria of the genus Listeria.

64. Method according to claim 60, characterized in that the target cells are selected from the group consisting of bacteria and bacterial spores, yeasts, fungi and fungal spores, plant cells and animal cells.

65. Method according to claim 60, characterized in that the cell wall binding polypeptide domains (CBD) are derived from the nucleotide sequence of (a) gene(s) and/or the amino acid sequence of (a) gene product(s) and are recovered therefrom.

66. Method according to claim 60, characterized in that the gene product(s) also comprise those gene products which are functional and effective only after post-translational modification.

67. Method according to claim 60, characterized in that the CBDs are directly bound to a detectable marker, preferably by genetic translational fusion.

68. Method according to claim 67, characterized in that the detectable marker is a fluorescent protein, preferably GFP, BFP, especially preferred GFP mut-1, GFP mut-2 or GFP mut-3, red fluorescence protein, cyan FP, Yellow FP.

69. Method according to claim 60, characterized in that the CBDs are directly bound with an amplifying substance which is detectable in further reactions, wherein the binding is preferably by genetic translational fusion.

70. Method according to claim 69, characterized in that the amplifying substance is biotin, peroxidase or phosphatase or another enzyme with a similar effect.

71. Method according to claim 60, characterized in that the CBDs are provided with detectable particulate markers, dyes, amplifying substances or isotopes.

72. Method according to claim 71 wherein the dye is a fluorescent dye.

73. Method according to claim 71 characterized in that the amplifying substance is biotin, peroxidase, phosphatase or another enzyme with a similar effect.

74. Method according to claim 60 wherein the CBD enable immobilization of the target cells to a solid surface by binding of the cell walls of the target cells and wherein said binding is carried out preferably at a pH between 7 and 10, more preferably a pH between 8 and 9 and an NaCl-content in the surrounding environment between 50 and 500 mM, preferably between 100 and 200 mM.

75. Method according to claim 60, characterized in that the target cells, immobilized by solid phase bound CBD are detected via a sandwich CBD assay with detectable and/or modified secondary CBD molecules.

76. Method according to claim 60, characterized in that the target cells immobilized by solid-phase bound CBD are detected via a sandwich-CBD-ELISA assay with detectable and/or modified secondary antibodies of the group of the immunoglobulins.

77. Method according to claim 60, characterized in that target cells immobilized by solid phase bound primary antibodies of the group of immunoglobulins are detected via a sandwich-IG-CBD assay with detectable and/or modified secondary CBD molecules.

78. Method according to claim 60, characterized in that CBD, bound to a mobile solid phase can bind target cells from a diluted and/or heterogenous mixture of cells and whereby in further steps the enrichment, isolation, purification and/or detection of said target cells is carried out.

79. Method according to claim 78, whereby the mobile phase consists of the magnetic beads as defined in claim 60.

80. Method according to claim 60, whereby the CBDs are CBD 500 and/or CBD 118.

81. Method according to claim 60, wherein the CBDs are CBD 500 and whereby the target cells are cells of the species Listeria monocytogenes Serovar 4,5 and/or 6.

82. Method according to claim 60, wherein the CBDs are CBD 118 and whereby the target cells are cells of the species Listeria monocytogenes Serovar ½, 3 and/or 7.

83. Method according to claim 60, whereby the CBDs are CBD 118 and whereby the target cells are growing cells of the species Listeria monocytogenes.

84. Method according to claim 60, whereby the binding of the target cells occurs via cell wall associated teichoic acids.

85. Method for the specific recognition of target cells by binding wherein the method comprises the following steps:
a) selection of proteins which specifically bind the target cells, whereby the target cells are bacteria of the genus Listeria,
b) provision of the protein domains which are responsible for the binding to the cell wall (CBD) as protein fragments, wherein these protein fragments do not have any hydrolytic activity, wherein the CBDs are CBD 500 and/or CBD 118,
c) covalent binding of the CBDs to a solid phase wherein the solid phase consists of magnetic latex beads which are preactivated with hydrophilic epoxy groups wherein the latex beads have an average surface of between 10 and 1000 μm²/bead, preferably between 10 and 100 μm²/bead, especially preferred between 20 and 50 μm²/bead and an average diameter of 1 to 40 μm, preferably 1 to 10 μm, especially preferred 2 to 5 μm,
d) contacting the CBDs as obtained according to step (c) with the sample to be examined, which comprises the target cells, and
e) optionally growing the target cells before or concurrently with steps (c), and/or (d).

86. Method according to claim 85, characterized in that the cell wall binding polypeptide domains (CBD) are derived from the nucleotide sequence of (a) gene(s) and/or the amino acid sequence of (a) gene product(s) and are recovered therefrom.

87. Method according to claim 85, characterized in that the gene product(s) also comprise those gene products which are functional and effective only after post-translational modification.

88. Method according to claim 85, characterized in that the CBDs are directly bound to a detectable marker, preferably by genetic translational fusion.

89. Method according to claim 88, characterized in that the detectable marker is a fluorescent protein, preferably GFP, BFP, especially preferred GFP mut-1, GFP mut-2 or GFP mut-3, red fluorescence protein, cyan FP, Yellow FP.

90. Method according to claim 85, characterized in that the CBDs are directly bound with an amplifying substance which is detectable in further reactions, wherein the binding is preferably by genetic translational fusion.

91. Method according to claim 90, characterized in that the amplifying substance is biotin, peroxidase or phosphatase or another enzyme with a similar effect.

92. Method according to claim 85, characterized in that the CBDs are provided with detectable particulate markers, dyes, amplifying substances or isotopes.

93. Method according to claim 92 wherein the dye is a fluorescent dye.

94. Method according to claim 92 characterized in that the amplifying substance is biotin, peroxidase, phosphatase or another enzyme with a similar effect.

95. Method according to claim 85 wherein the CBD enable immobilization of the target cells to a solid surface by binding of the cell walls of the target cells and wherein said binding is carried out preferably at a pH between 7 and 10, more preferably a pH between 8 and 9 and an NaCl-content in the surrounding environment between 50 and 500 mM, preferably between 100 and 200 mM.

96. Method according to claim 85, characterized in that the target cells, immobilized by solid phase bound CBD are detected via a sandwich CBD assay with detectable and/or modified secondary CBD molecules.

97. Method according to claim 85, characterized in that the target cells immobilized by solid-phase bound CBD are detected via a sandwich-CBD-ELISA assay with detectable and/or modified secondary antibodies of the group of the immunoglobulins.

98. Method according to claim 85, characterized in that target cells immobilized by solid phase bound primary antibodies of the group of immunoglobulins are detected via a sandwich-IG-CBD assay with detectable and/or modified secondary CBD molecules.

99. Method according to claim 85, characterized in that CBD, bound to a mobile solid phase can bind target cells from a diluted and/or heterogenous mixture of cells and whereby in further steps the enrichment, isolation, purification and/or detection of said target cells is carried out.

100. Method according to claim 99, wherein the mobile phase consists of the magnetic beads as defined in claim 85.

101. Method according to claim 85, wherein the CBDs are CBD 500 and whereby the target cells are cells of the species Listeria monocytogenes Serovar 4, 5 and/or 6.

102. Method according to claim 85, wherein the CBDs are CBD 118 and whereby the target cells are cells of the species Listeria monocytogenes Serovar ½, 3 and/or 7.

103. Method according to claim 85, wherein the CBDs are CBD 118 and whereby the target cells are growing cells of the species Listeria monocytogenes.

104. Method according to claim 85, wherein the binding of the target cells occurs via cell wall associated teichoic acids.

105. Method for the specific recognition of target cells by binding wherein the method comprises the following steps:
a) selection of proteins which specifically bind the target cells, whereby the target cells are bacteria of the genus Listeria,
b) provision of the protein domains which are responsible for the binding to the cell wall (CBD) as protein fragments, wherein these protein fragments do not have any hydrolytic activity, wherein the CBDs are CBD 500 and/or CBD 118,
c) covalent binding of the CBDs to a solid phase wherein the solid phase consists of magnetic latex beads which are preactivated with hydrophilic epoxy groups wherein the latex beads have an average surface of between 10 and 1000 μm²/bead, preferably between 10 and 100 μm²/bead, especially preferred between 20 and 50 μm²/bead and an average diameter of 1 to 40 μm, preferably 1 to 10 μm, especially preferred 2 to 5 μm,
d) contacting the CBDs as obtained according to step (c) with the sample to be examined, which comprises the target cells, and
e) optionally growing the target cells before or concurrently with steps (c), and/or (d).
wherein the CBDs are directly bound to a detectable marker, whereby the marker is green fluorescent protein (GFP, especially GFP/mut-1, GFP/mut-2 or GFP/mut-3), red fluorescence protein, cyan FP, Yellow FP, and whereby the GFP provides the binding between the CBD and the solid phase.

106. Method according to claim 105, characterized in that the cell wall binding polypeptide domains (CBD) are derived from the nucleotide sequence of (a) gene(s) and/or the amino acid sequence of (a) gene product(s) and are recovered therefrom.

107. Method according to claim 105, characterized in that the gene product(s) also comprise those gene products which are functional and effective only after post-translational modification.

108. Method according to claim 105, characterized in that the CBDs are directly bound to the detectable marker by genetic translational fusion.

109. Method according to claim 105 wherein the CBD enable immobilization of the target cells to a solid surface by binding of the cell walls of the target cells and wherein said binding is carried out preferably at a pH between 7 and 10, more preferably a pH between 8 and 9 and an NaCl-content in the surrounding environment between 50 and 500 mM, preferably between 100 and 200 mM.

110. Method according to claim 105, characterized in that the target cells, immobilized by solid phase bound CBD are detected via a sandwich CBD assay with detectable and/or modified secondary CBD molecules.

111. Method according to claim 105, characterized in that the target cells immobilized by solid-phase bound CBD are detected via a sandwich-CBD-ELISA assay with detectable and/or modified secondary antibodies of the group of the immunoglobulins.

112. Method according to claim 105, characterized in that the target cells immobilized by solid phase bound primary antibodies of the group of immunoglobulins are detected via a sandwich-IG-CBD assay with detectable and/or modified secondary CBD molecules.

113. Method according to claim 105, characterized in that CBD, bound to a mobile solid phase, can bind target cells from a diluted and/or heterogenous mixture of cells and whereby in further steps the enrichment, isolation, purification and/or detection of said target cells is carried out.

114. Method according to claim 113, wherein the mobile phase consists of the magnetic beads as defined in claim 105.

115. Method according to claim 105, wherein the CBDs are CBD 500 and whereby the target cells are cells of the species Listeria monocytogenes Serovar 4, 5 and/or 6.

116. Method according to claim 105, wherein the CBDs are CBD 118 and whereby the target cells are cells of the species Listeria monocytogenes Serovar ½, 3 and/or 7.

117. Method according to claim 105, wherein the CBDs are CBD 118 and whereby the target cells are growing cells of the species Listeria monocytogenes.

118. Method according to claim 105, wherein the binding of the target cells occurs via cell wall associated teichoic acids.

119. Use of the method according to one of claims 1 to 118 for detection, diagnosis, immobilization or enrichment of cells.

120. Reagent kit for a method according to one of claims 1 to 118, comprising additionally to conventional detection means one or more CBDs, obtained according to step (b) as defined claim 1, bound as defined in step (c) of claim 1.

121. Biochip comprising a CBD as defined above.

122. Biochip according to claim 121, wherein the biochip is a BIA core or SELDI biochip.

123. Biochip according to claim 121 wherein it comprises two or more different CBDs on defined locations.
